(19) United States
(12) Patent Application Publication
(10) Pub. No.: US 2014/0350865 A1
(43) Pub. Date: Nov. 27, 2014

(54) CONTENT INFORMATION OUTPUT

(71) Applicant: Nec Biglobe, Ltd., Tokyo (JP)

(72) Inventor: Michitaro MIYATA, Tokyo (JP)

(73) Assignee: Nec Biglobe, Ltd., Tokyo (JP)

(21) Appl. No.: 14/263,004

(22) Filed: Apr. 28, 2014

(30) Foreign Application Priority Data
May 27, 2013 (JP) 2013-110624

(51) Int. Cl. A61B 5/00; A61B 5/024

(52) U.S. Cl. CPC A61B 5/7282; A61B 5/024; A61B 5/742; A61B 5/741; A61B 5/7475; A61B 5/681
USPC 702/19

(57) ABSTRACT

A communication terminal obtains biological information. The communication terminal determines whether the obtained biological information satisfies preset conditions. When it is determined that the biological information satisfies preset conditions, the communication terminal creates content information indicating the contents of the output information. The communication terminal then provides the created content information to other communication terminals.

This application is based upon and claims the benefit of priority from Japanese Patent Application No. 2013-110624 filed on May 27, 2013, the content of which is incorporated by reference.

### TECHNICAL FIELD

The present invention relates to an information processing system for performing information processing, an information processing terminal, an information processing method, and a program.

### BACKGROUND ART

Recently, on a communication network, there is information considered unnecessary for maintaining healthy life such as distribution of contents unsuitable for children to view or electronic mail describing malicious contents. When a child views such information, the child finds it hard to report it to his parents, and the parents are not aware of such a situation. Thus, the parents cannot prevent the child from accessing such information.

Under these circumstances, a technology for placing restrictions on sites other than those registered beforehand by a person in parental authority or the like of a child to deny access to the child has been invented (e.g., refer to Patent Literature 1).

### CITATION LIST

### Patent Literature

Patent Literature 1: JP2003-186842A

### SUMMARY OF INVENTION

### Technical Problem

Such a technology has a problem of time and labor necessary for registration. The technology also has a problem, i.e., when inappropriate information is present in a part of even the registered sites, the parents cannot prevent the child from accessing such information.

It is therefore an object of the present invention to provide an information processing system, an information processing terminal, an information processing method, and a program that can solve the aforementioned problems.

### Solution to Problem

According to the present invention, there is provided an information processing system including:

a biological information measurement unit that measures biological information;

a first information processing terminal that includes a first output unit configured to output information, and a biological information acquisition unit configured to acquire the biological information measured by the biological information measurement unit from the biological information measurement unit;

a second information processing terminal; and

a condition determination unit that determines whether or not the biological information acquired by the biological information acquisition unit satisfies preset conditions.

In this case, the first information processing terminal includes a content information creation unit configured to create, when the condition determination unit determines that the biological information satisfies the conditions, content information indicating contents of the information output from the first output unit; and

a content information providing unit that provides the content information created by the content information creation unit to the second information processing terminal.

The second information processing terminal includes a second output unit configured to output information indicated by the content information provided from the content information providing unit.

According to the present invention, there is provided an information processing terminal including:

an output unit that outputs information;

a biological information acquisition unit that acquires biological information;

a condition determination unit that determines whether or not the biological information acquired by the biological information acquisition unit satisfies preset conditions;

a content information creation unit that creates, when the condition determination unit determines that the biological information satisfies the conditions, content information indicating contents of the information output from the output unit; and

a content information providing unit that provides the content information created by the content information creation unit.

According to the present invention, there is provided an information processing method including:

processing for outputting information;

processing for acquiring biological information;

processing for determining whether the acquired biological information satisfies preset conditions;

processing for creating, when the biological information satisfies the conditions, content information indicating contents of the output information; and

processing for providing the created content information.

According to the present invention, there is provided a program for causing a computer to execute:

a procedure for outputting information;

a procedure for acquiring biological information;

a procedure for determining whether or not the acquired biological information satisfies preset conditions;

a procedure for creating, when the biological information satisfies the conditions, content information indicating contents of the output information; and

a procedure for providing the created content information.

### Advantageous Effects of Invention

As described above, according to the present invention, a user can know that inappropriate information has been output.

The above and other objects, features, and advantages of the present invention will become apparent from the following description with reference to the accompanying drawings which illustrate an example of the present invention.

### BRIEF DESCRIPTION OF DRAWINGS

FIG. 1 is a diagram showing an information processing system according to the first embodiment of the present invention;

FIG. 2 is a diagram showing an example of the internal configuration of a communication terminal operated by a viewer shown in FIG. 1;

FIG. 3 is a diagram showing an example of a biological information table stored in a storage unit shown in FIG. 2;

FIG. 4 is a diagram showing an example of a threshold value table stored in the storage unit shown in FIG. 2;

FIG. 5 is a diagram showing an example of a content information table stored in the storage unit shown in FIG. 2;

FIG. 6 is a diagram showing an example of the threshold value table stored in the storage unit 180 shown in FIG. 2, where time information is added;

FIG. 7 is a diagram showing an example of the content information table stored in the storage unit 180 shown in FIG. 2, where time information is added;

FIG. 8 is a diagram showing an example of the internal configuration of the communication terminal operated by a checker shown in FIG. 1;

FIG. 9 is a flowchart illustrating an example of processing to create content information in the communication terminal operated by the viewer shown in FIG. 1;

FIG. 10 is a sequential diagram illustrating an example of processing when content information is requested from the communication terminal operated by the checker shown in FIG. 1;

FIG. 11 is a sequential diagram illustrating another example of processing when the content information is requested from the communication terminal operated by the checker shown in FIG. 1;

FIG. 12 is a diagram showing an information processing system according to the second embodiment of the present invention;

FIG. 13 is a diagram showing an example of the internal configuration of a communication terminal operated by a viewer shown in FIG. 12;

FIG. 14 is a diagram showing an example of the internal configuration of a management server shown in FIG. 12;

FIG. 15 is a diagram showing an example of a user related table stored in a storage unit shown in FIG. 14;

FIG. 16 is a sequential diagram illustrating an example of processing to create content information in the communication terminal operated by the viewer and the management server shown in FIG. 12;

FIG. 17 is a sequential diagram illustrating another example of processing when content information is requested from the communication terminal operated by a checker shown in FIG. 12;

FIG. 18 is a diagram showing an information processing system according to the third embodiment of the present invention;

FIG. 19 is a diagram showing an example of the internal configuration of a communication terminal operated by a checker shown in FIG. 18;

FIG. 20 is a sequential diagram illustrating an example of processing to create content information in the communication terminal operated by a viewer shown in FIG. 18;

FIG. 21 is a flowchart illustrating an example of processing to output the content information in the communication terminal operated by the checker shown in FIG. 18;

FIG. 22 is a diagram showing an information processing system according to the fourth embodiment of the present invention;

FIG. 23 is a diagram showing an example of the internal configuration of a communication terminal operated by a viewer shown in FIG. 22;

FIG. 24 is a diagram showing an example of the internal configuration of a communication terminal operated by a checker shown in FIG. 22;

FIG. 25 is a sequential diagram illustrating an example of processing to create content information in the communication terminal shown in FIG. 22;

FIG. 26 is a flowchart illustrating an example of processing to output the content information in the communication terminal operated by the checker shown in FIG. 22;

FIG. 27 is a diagram showing an information processing system according to the fifth embodiment of the present invention;

FIG. 28 is a diagram showing an example of the internal configuration of a management server shown in FIG. 27;

FIG. 29 is a sequential diagram illustrating an example of processing to create content information in a communication terminal operated by a viewer and the management server shown in FIG. 27;

FIG. 30 is a sequential diagram illustrating an example of processing to output the content information in the management server and the communication terminal operated by a checker shown in FIG. 27;

FIG. 31 is a diagram showing an information processing system according to the sixth embodiment of the present invention;

FIG. 32 is a diagram showing an information processing system according to the seventh embodiment of the present invention;

FIG. 33 is a diagram showing an information processing system according to the eighth embodiment of the present invention;

FIG. 34 is a diagram showing an information processing system according to the ninth embodiment of the present invention;

FIG. 35 is a sequential diagram illustrating an example of processing to create content information in a communication terminal shown in FIG. 34;

FIG. 36 is a diagram showing an information processing system according to the tenth embodiment of the present invention; and

FIG. 37 is a sequential diagram illustrating an example of processing to create content information in a communication terminal operated by a viewer and a management server shown in FIG. 36.

### EXEMPLARY EMBODIMENTS

Hereinafter, the embodiments of the present invention will be described with reference to the drawings.

### First Embodiment

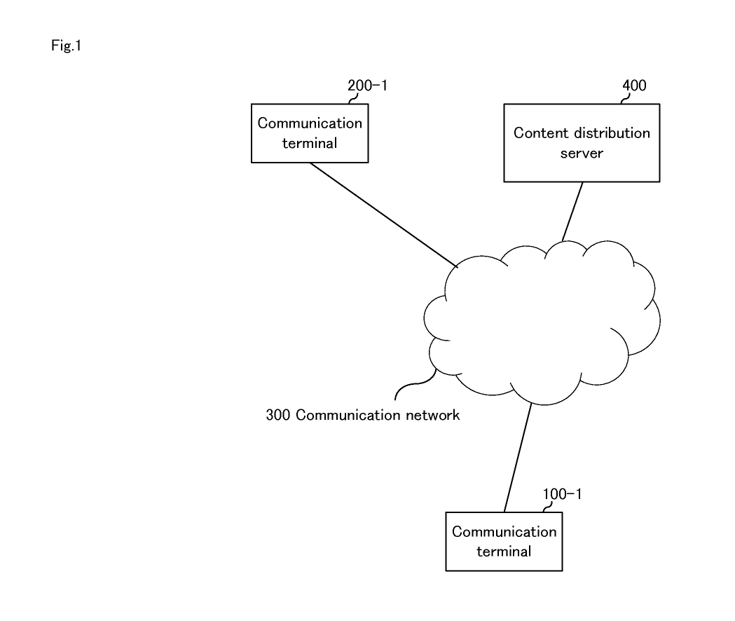

FIG. 1 shows an information processing system according to the first embodiment of the present invention, where communication terminals 100-1 and 200-1 and content distribution server 400 can communicate with each other via communication network 300. In the exemplary embodiment shown in FIG. 1, one communication terminal 100-1, one communication terminal 200-1, and one content distribution server 400 are shown. However, there are no restrictions on the number of terminals and servers thereof.

Communication terminal 100-1 is a first information processing terminal operated by a content viewing monitoring subject (hereinafter, referred to as viewer) such as a child or a student (pupil).

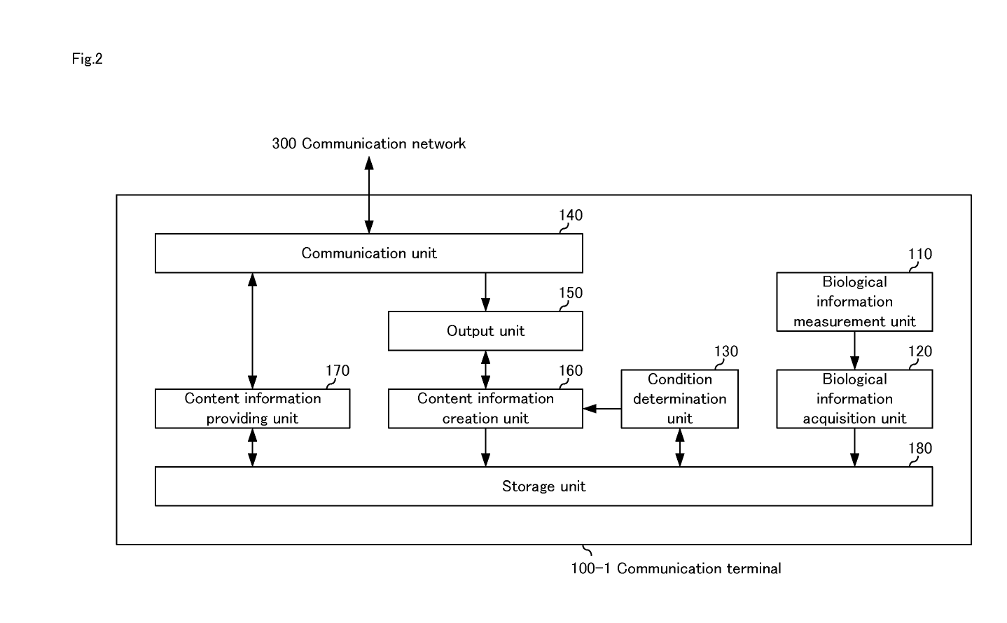

Communication terminal 100-1 shown in FIG. 1 includes, as shown in FIG. 2, biological information measurement unit 110, biological information acquisition unit 120, condition determination unit 130, communication unit 140, output unit 150, content information creation unit 160, content information providing unit 170, and storage unit 180. FIG. 2 shows an example of the main components, according to the embodiment, from among the components arranged in communication terminal 100-1 shown in FIG. 1.

Biological information measurement unit 110 measures the biological information of a user operating communication unit 100-1. The reason the biological information is measured is because the present invention uses a change in biological information such as a pulse rate during viewing and listening when the user who is operating communication terminal 100-1, and who is a content viewer, views abnormal contents. The biological information measured by biological information measurement unit 110 is the physiological information of the user easily changed according to a stimulus from the outside, such as the pulse rate, the heart rate, the amount of perspiration, the brain wave, the vocal print (voice tone), or the amount of secreted adrenaline of the user operating communication terminal 100-1.

One example will be described as a method for measuring such biological information by using biological information measurement unit 110. For example, when the pulse rate is used as biological information, and a pulse rate per 60 seconds is a measured value, biological information measurement unit 110 detects the pulse rate of the viewer for 60 seconds from the start of measurement, and transmits the pulse fate detected during the period to biological information acquisition unit 120. Then, biological information acquisition unit 120 sets the acquired pulse rate as a measured value. In the present invention, to facilitate specifying of the place of contents viewed by the viewer, biological information measurement unit 110 can detect the pulse rate of the viewer for a predetermined short period (e.g., 5 seconds) or a predetermined pulse rate (e.g., 5 rates) from the start of measurement, and transmit the pulse rate detected during the period or time to biological information acquisition unit 120. Then, biological information acquisition unit 120 can predict a pulse rate per 60 seconds from the acquired pulse rate or time, and set the predicted pulse rate per 60 seconds as a measured value. Biological information measurement unit 110 can directly transmit the waveform data of a pulse rate to biological information acquisition unit 120 after the start of detecting the pulse rate of the viewer. Biological information acquisition unit 120 can temporarily store the acquired waveform data for a predetermined period (e.g., 5 minutes), and calculate a pulse rate per 60 seconds by using past (e.g., 5 second or 60 second before) waveform data at predetermined timing (e.g., interval of 5 seconds or 5 rates) to set the pulse rate as a measured value. However, the method for measuring the biological information by biological information measurement unit 110 can be a general method implemented at a medical institution.

The timing for measuring the biological information by biological information measurement unit 110, which may be timing for starting the measurement or timing for transmitting the biological information to biological information acquisition unit 120 depending on the method for measuring the biological information, can be timing that uses a preset period or the transition of a display screen. However, when elapsed time is used for determination at condition determination unit 130 described below, the timing (period) for measuring the biological information by biological information measurement unit 110, which is shorter than the elapsed time, must be used. According to the method for measuring the biological information, the timing for starting detection of the biological information from the viewer and the timing for starting measurement of the biological information detected by biological information measurement unit 110 can be simultaneous, or the measurement can be started at preset timing after the detection of the biological information.

Biological information measurement unit 110 is attached to viewer operating communication terminal 100-1 to measure the biological information. The shape of communication terminal 100-1 including biological information measurement unit 110 is a wristwatch type, a wristband type, or a glove type where a attaching unit such as a belt, a wristband, or a glove is fixed to the casing of communication terminal 100-1, and the casing is attached to the viewer. For example, when the shape of communication terminal 100-1 is a wristwatch type, biological information measurement unit 110 is disposed on communication terminal 100-1's casing side that is next to the skin of the viewer. It is only necessary for biological information measurement unit 110 to be formed into a shape suitable for measuring the biological information that is a measuring target.

Biological information measurement unit 110 can include a sensor for detecting whether it is attached to the viewer, and measure the biological information only when the measurement unit is attached to the viewer. Biological information measurement unit 110 can include a switch that can be set to validate or invalidate a measurement function from the outside, and can measure the biological information only when the switch is set to validate the measurement function. Biological information measurement unit 110 can measure the biological information only when output unit 150 outputs (displays) contents.

Biological information acquisition unit 120 acquires the biological information measured by biological information measurement unit 110. Biological information acquisition unit 120 writes the acquired biological information in storage unit 180. Biological information acquisition unit 120 can calculate a measured value from the acquired biological information by using a predetermined calculation method, and then write the measured value in storage unit 180. If biological information has been written in storage unit 180, biological information acquisition unit 120 can write the acquired biological information over it, or add the acquired biological information in association with time.

Condition determination unit 130 determines whether the biological information stored in storage unit 180 satisfies preset conditions. The conditions have been stored beforehand in storage unit 180. When the biological information stored in storage unit 180 exceeds a preset threshold value, condition determination unit 130 determines that the biological information satisfies the conditions. Condition determination unit 130 can determine that the biological information satisfies the conditions when a state where the biological information stored in storage unit 180 exceeds the preset threshold value continues for a predetermined time.

Biological information acquisition unit 120 can not write all pieces of acquired biological information in storage unit 180 but only those determined to satisfy the conditions by condition determination unit 130 in storage unit 180.

Communication unit 140 has a communication interface function for performing communication with communication network 300.

Output unit 150 outputs information (first output unit). For example, output unit 150 can output information in the form of displaying the information, or as a voice. Contents output from output unit 150 can be temporarily stored in storage unit 180 for a predetermined time (e.g., time from measurement start of biological information to the end or 5 minutes).

Content information creation unit 160 creates content information indicating the contents of the information output from output unit 150 when condition determination unit 130 determines that the biological information satisfies the conditions. The content information is a capture screen capturing a screen currently displayed by output unit 150, the main text information of electronic mail currently displayed by output unit 150, the file and the file name of moving image data currently displayed by output unit 150, the file and the file name of some still image data among the moving image data currently displayed by output unit 150, or information indicating the URL (Uniform Resources Locator) of a site connected to acquire the screen currently displayed by output unit 150. In communication terminal 200-1, based on the content information, the contents that have been viewed by the viewer by operating communication terminal 100-1 can be recognized. Content information creation unit 160 writes the created content information in storage unit 180. If the contents output from output unit 150 have been temporarily stored in storage unit 180 for a predetermined time, content information creation unit 160 can create content information from all or some of the temporarily stored contents, and then write the content information in storage unit 180.

Content information providing unit 170 provides (transmits), by using communication unit 140, the content information stored in storage unit 180 to communication terminal 200-1 via communication network 300. Content information providing unit 170 can store the transmission history of content information to communication terminal 200-1, but can not transfer any content information that have been transmitted to communication terminal 100-1. The content information provided (transmitted) to communication terminal 200-1 by content information providing unit 170 can be limited to content information created during a predetermined period and stored.

Storage unit 180 stores information such as the preset threshold value, the biological information written by biological information acquisition unit 120, or the content information written by content information creation unit 160.

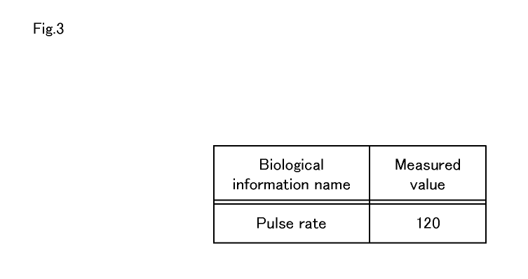

In the biological information table stored in storage unit 180 shown in FIG. 2, as shown in FIG. 3, a biological information name and a measured value are associated with each other. The biological information name is information indicating the type of biological information measured by biological information measurement unit 110. The measured value is a value using the biological information measured by biological information measurement unit 110. In the biological information table, a viewer identifier indicating the viewer of the measured value is stored in association.

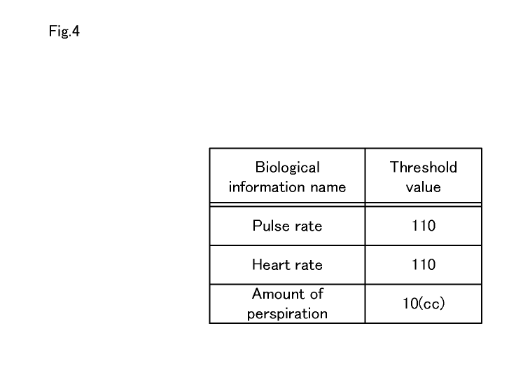

In the threshold value table stored in storage unit 180 shown in FIG. 2, as shown in FIG. 4, a biological information name and a threshold value are associated with each other. The threshold value is information used as a reference for determination at condition determination unit 130. For example, when the threshold value table shown in FIG. 4 is stored in storage unit 180 and the biological information table shown in FIG. 3 is stored in storage unit 180, a pulse rate “120” stored in the biological information table exceeds the pulse rate threshold value “110” stored in the threshold value table. Thus, condition determination unit 130 determines that the biological information satisfies the conditions.

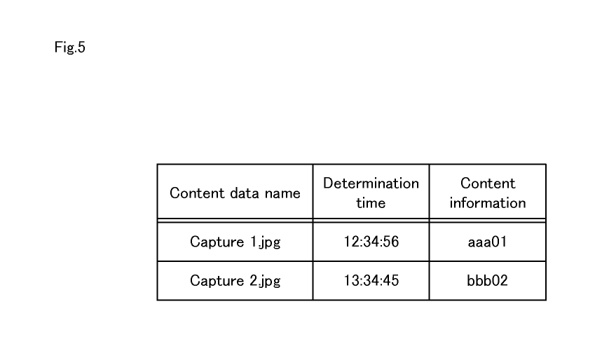

In the content information table stored in storage unit 180 shown in FIG. 2, as shown in FIG. 5, a content data name, determination time, a content information associated with a viewer identifier are stored. The determination time is information indicating the time of determining satisfaction of the conditions by condition determination unit 130. The content data name is information (e.g., file name) indicating capture screen information capturing a screen output (displayed) by output unit 150. The viewer identifier is the identification information of a viewer operating communication terminal 100-1 at the determined time. The content information can be information including no viewer identifier. When a plurality of users uses communication terminal 100-1, viewer identifiers input by the respective users to log in by using communication terminal 100-1 can be added to the content information.

Time information can be added to the threshold value table, and an example will be described below.

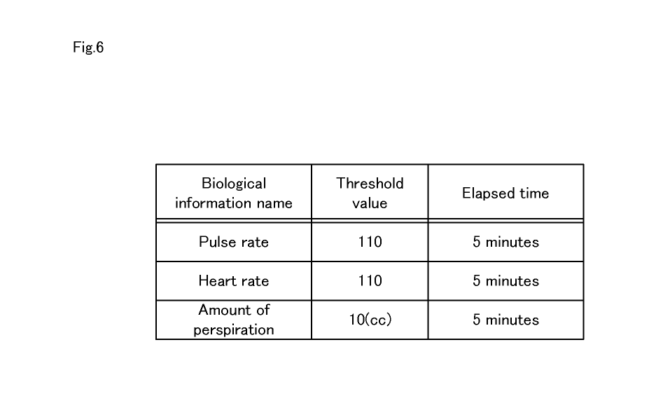

In the threshold value table to which time information has been added and which has been stored in storage unit 180 shown in FIG. 2, as shown in FIG. 6, elapsed time is associated in addition to the biological information name and the threshold value. For example, when the threshold value table shown in FIG. 6 is stored in storage unit 180, condition determination unit 130 determines that the biological information satisfies the conditions if a state where a pulse rate stored in the biological information table exceeds a pulse rate threshold value stored in the threshold value table continues for 5 minutes or longer.

Time information indicating determination of satisfying of the conditions by condition determination unit 130 can be added to the content information table, and an example will be described below.

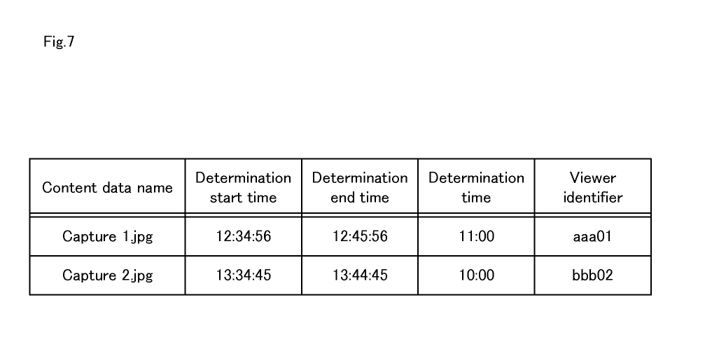

In the content information table to which time information has been added and which has been stored in storage unit 180 shown in FIG. 2, as shown in FIG. 7, content information that associates a content data name, determination start time, determination end time, determination time, and a viewer identifier with one another is stored. The determination start time is information indicating the time of a change from a state where condition determination unit 130 determines that the biological information does not satisfy the conditions to a state where it determines that the biological information satisfies the conditions. The determination end time is information indicating the time of a change from a state where condition determination unit 130 determines that the biological information satisfies the conditions to a state where it determines that the biological information does not satisfy the conditions. The determination time indicates time from the determination start time to the determination end time. The determination time is used when condition determination unit 130 executes determination by using the elapsed time.

Communication terminal 200-1 is a second information processing terminal operated by a monitor (hereinafter, referred to as checker) such as parents or a teacher monitoring contents viewed by communication terminal 100-1.

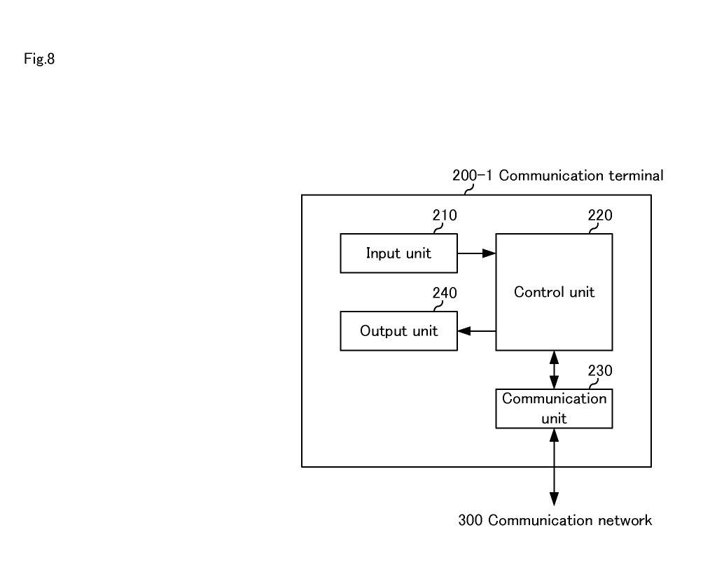

Communication terminal 200-1 shown in FIG. 1 includes, as shown in FIG. 8, input unit 210, control unit 220, communication unit 230, and output unit 240. FIG. 8 shows an example of the main components according to the embodiment among the components arranged in communication terminal 200-1 shown in FIG. 1.

Input unit 210 receives information input from the outside. Input unit 210 can be configured by hardware such as an input key button using a predetermined key or a touch panel.

Control unit 220 requests, by using communication unit 230, after input unit 210 has received a predetermined input, communication terminal 100-1 to acquire content information via communication network 300. Control unit 220 causes, after the content information has been provided (transmitted) from communication terminal 100-1 via communication network 300, output unit 240 to output information indicated by the content information.

Communication unit 230 has a communication interface function for performing communication with communication network 300.

Output unit 240 outputs information according to an instruction from control unit 220 (second output unit). For example, output unit 240 can output information in the form of displaying information, or as a voice.

Content distribution server 400 is a server that distributes contents to communication terminal 100-1. The contents include one from among a still image or a moving image, a multimedia content similar to an application, electronic mail or a novel, a comment on an electronic bulletin board and a text content similar to a message of a message exchange service. The contents distributed by content distribution server 400 can be downloaded from communication terminal 100-1. If the content information is URL or the like, and that contents that have been viewed by the viewer cannot be recognized based only on the content information at communication terminal 200-1, content distribution server 400 can be accessed from communication terminal 200-1 by using the content information, and the contents can be redistributed or downloaded from content distribution server 400.

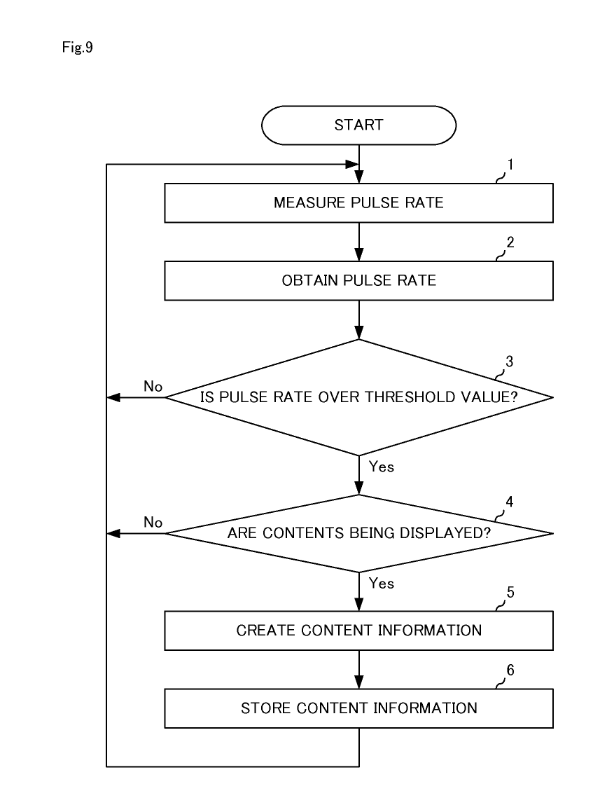

Hereinafter, an information processing method in the embodiment shown in FIG. 1 will be described. First, the processing to create content information at communication terminal 100-1 shown in FIG. 1 will be described referring to FIG. 9. An example where biological information is a pulse rate will be described. An example where output unit 150 outputs content by displaying the content will be described.

First, in step 1, biological information measurement unit 110 attached to the viewer operating communication terminal 100-1 measures the pulse rate of the viewer by a predetermined measurement method and at predetermined timing. In step 2, biological information acquisition unit 120 acquires the pulse rate measured by biological information measurement unit 110 from biological information measurement unit 110. Then, biological information acquisition unit 120 writes and stores the acquired pulse rate in the biological information table of storage unit 180 after calculation, if necessary.

In step 3, condition determination unit 130 determines whether the pulse rate stored in the biological information table of storage unit 180 exceeds the threshold value preset in the threshold value table of storage unit 180. When condition determination unit 130 determines that the pulse rate stored in the biological information table of storage unit 180 exceeds the threshold value preset in the threshold value table of storage unit 180, in step 4, content information creation unit 160 determines whether output unit 150 is displaying the contents.

When content information creation unit 160 determines that output unit 150 is displaying the contents, in step 5, content information creation unit 160 creates content information indicating the contents being displayed, and in step 6, writes and stores the content information in the content information table of storage unit 180.

On the other hand, when condition determination unit 130 determines that the pulse rate stored in the biological information table of storage unit 180 does not exceed the threshold value preset in the threshold value table of storage unit 180 in step 3, or content information creation unit 160 determines that output unit 150 is not displaying the contents in step 4, the processing of step 1 is executed at next timing.

In step 1, whether or not output unit 150 is displaying the contents is determined. When content information creation unit 160 determines that output unit 150 is displaying the contents, the pulse rate of the viewer can be measured. In this case, in steps 2, 3, and 5 (not step 4), when condition determination unit 130 determines that the pulse rate stored in the biological information table of storage unit 180 exceeds the threshold value preset in the threshold value table of storage unit 180, content information creation unit 160 only needs to create content information indicating that the contents are being displayed.

For example, in the case of biological information needing certain measurement time of a pulse rate or a heat rate or detected data, the continuously measured biological information is acquired, and then the process can be divided into preprocessing for writing the acquired biological information as the continuous data of a predetermined period of time (e.g., waveform data) in storage unit 180 by biological information acquisition unit 120 after step 2 and post-processing for determining whether biological information satisfies the conditions by using the continuous data of the biological information written in storage unit 180 after step 3. In this case, when condition determination unit 130 determines that the threshold value is not exceeded in step 3, or content information creation unit 160 determines that the contents are not being displayed in step 4, the processing of step 3 is executed at next timing after step 6. The post-processing can be executed at a predetermined timing different from that of the preprocessing.

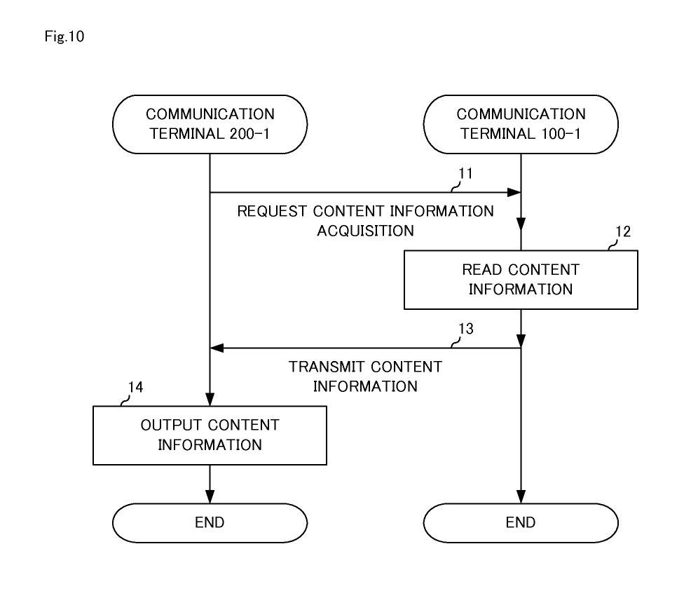

Next, processing when content information is requested from communication terminal 200-1 shown in FIG. 1 will be described referring to FIG. 10.

After input unit 210 of communication terminal 200-1 has received a predetermined input, in step 11, control unit 220 requests acquisition of content information to communication terminal 100-1 via communication network 300 by using communication unit 230. For this request, a request signal enabling recognition of the request to acquire content information at communication terminal 100-1 can be used.

After a request has been made to acquire content information from communication terminal 200-1, in step 12, content information providing unit 170 reads the content information stored in the content information table of storage unit 180. Then, in step 13, content information providing unit 170 transmits the read content information to communication terminal 200-1 via communication network 300 by using communication unit 140.

After communication unit 230 has received the content information transmitted from communication terminal 100-1, in step 14, control unit 220 outputs information indicated by the content information from output unit 240. This information can be a capture screen when the content information includes the capture screen itself, or a URL when the content information includes the URL of a content distribution site.

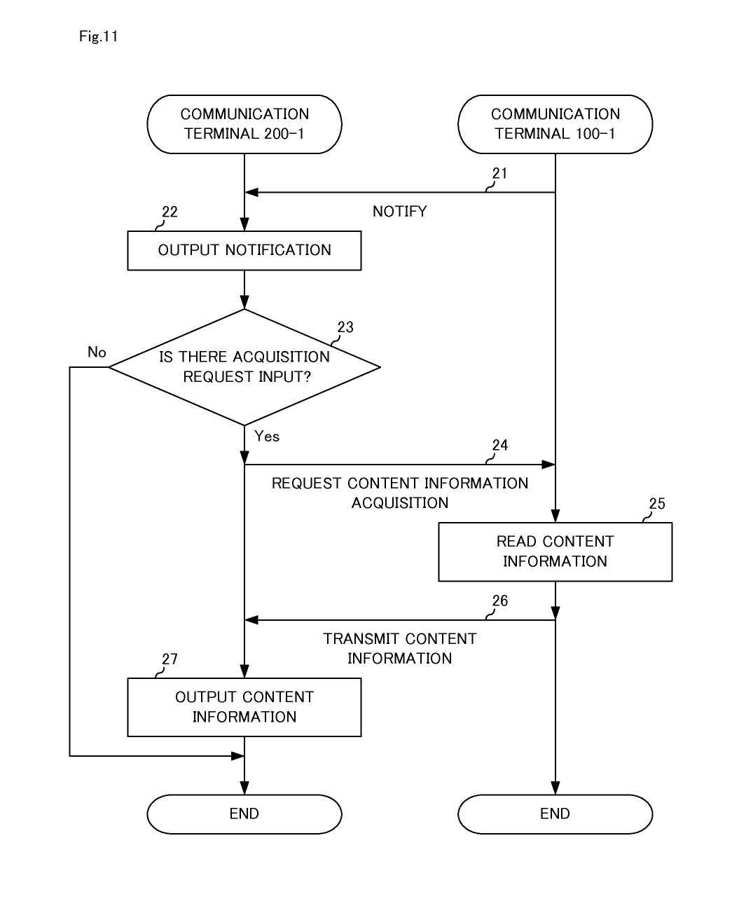

After communication terminal 100-1 has created the content information, its message can be notified to communication terminal 200-1. An example of processing in this case will be described by referring to FIG. 11.

After content information creation unit 160 of communication terminal 100-1 has created content information and written it in storage unit 180, in step 21, content information providing unit 170 issues a predetermined notification to communication terminal 200-1 by using communication unit 140. This notification can be any type as long as communication terminal 200-1 can recognize the creation of the content information by using, e.g., electronic mail. After communication unit 230 of communication terminal 200-1 has received notification from communication terminal 100-1, in step 22, control unit 220 outputs its message from output unit 240. For example, control unit 220 displays a message indicating creation of the content information on display unit 240.

Then, in step 23, control unit 220 determines whether input unit 210 of communication terminal 200-1 has received a predetermined input. When control unit 220 determines that input unit 210 of communication terminal 200-1 has received the predetermined input, in step 24, control unit 220 requests acquisition of content information to communication terminal 100-1 via communication network 300 by using communication unit 230.

After the acquisition of the content information has been requested from communication terminal 200-1, in step 25, content information providing unit 170 reads the content information stored in the content information table of storage unit 180. Then, in step 26, content information providing unit 170 transmits the read content information to communication terminal 200-1 via communication network 300 by using communication unit 140.

After communication unit 230 has received the content information transmitted from communication terminal 100-1, in step 27, control unit 220 outputs information indicated by the content information from output unit 240.

### Second Embodiment

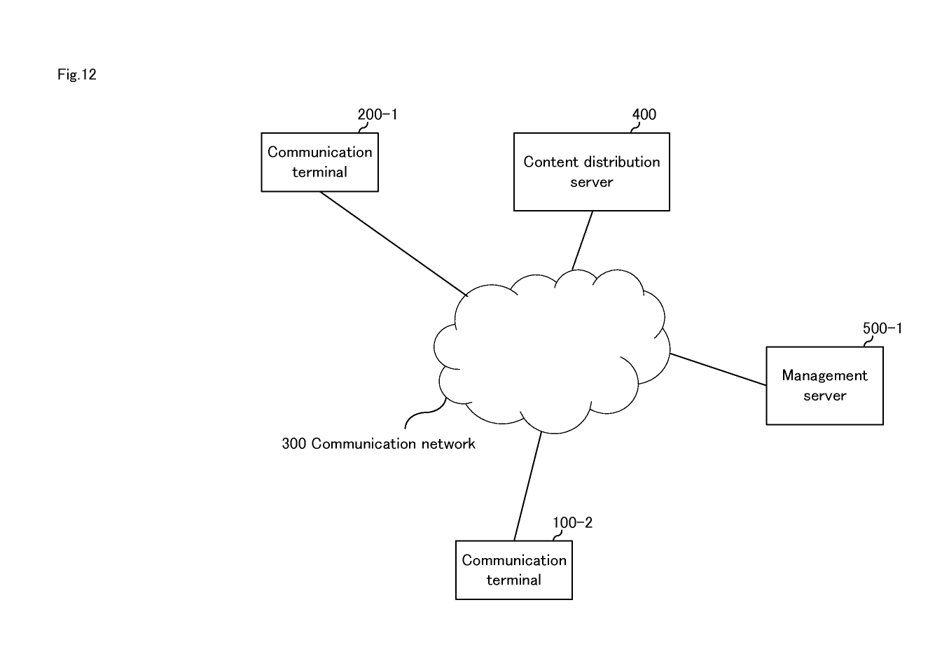

FIG. 12 shows an information processing system according to the second embodiment of the present invention, where communication terminals 100-2 and 200-1, content distribution server 400, and management server 500-1 can communicate with one another via communication network 300. In the exemplary embodiment shown in FIG. 12, one communication terminal 100-2, one communication terminal 200-1, and one content distribution server 400 are shown. However, there are no restrictions on the numbers thereof. In the second embodiment, content information created by communication terminal 100-2 is stored by management server 500-1.

Communication terminal 200-1 and content distribution server 400 are similar to those shown in FIG. 1.

Communication terminal 100-2 is a first information processing terminal operated by a viewer.

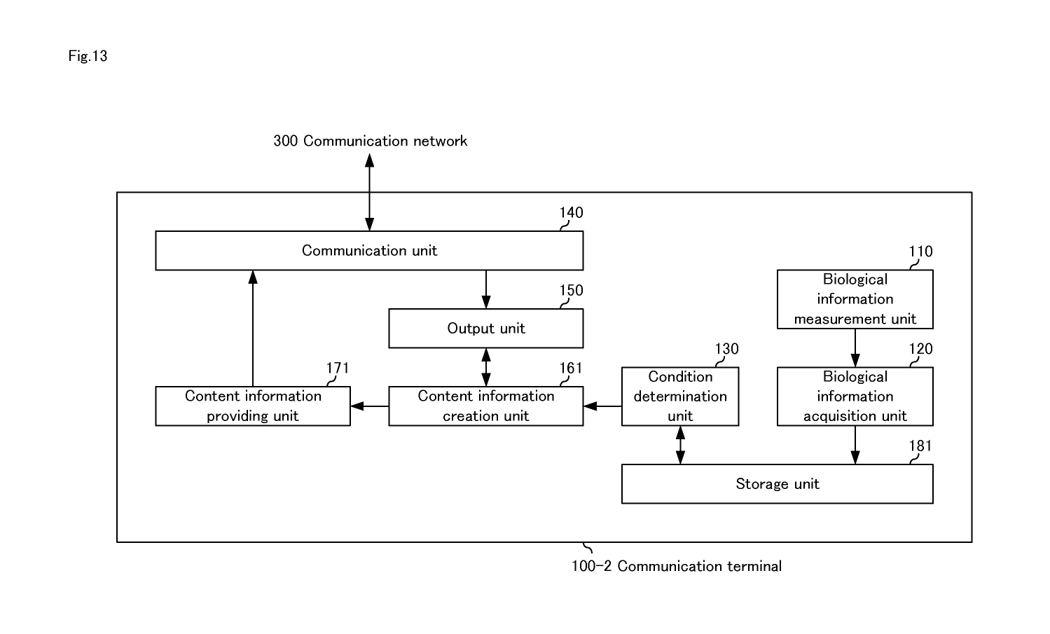

Communication terminal 100-2 shown in FIG. 12 includes, as shown in FIG. 13, biological information measurement unit 110, biological information acquisition unit 120, condition determination unit 130, communication unit 140, output unit 150, content information creation unit 161, content information providing unit 171, and storage unit 181. FIG. 13 shows an example of main components according to the embodiment among the components arranged in communication terminal 100-2 shown in FIG. 12.

Biological information measurement unit 110, biological information acquisition unit 120, condition determination unit 130, communication unit 140, output unit 150 are similar to those shown in FIG. 2.

Content information creation unit 161 creates content information as in the case of content information creation unit 160 shown in FIG. 2. However, content information creation unit 161 outputs the created content information to content information providing unit 170 different from the case of content information creation unit 160 that writes the created content information in the storage unit.

Content information providing unit 171 transmits, by using communication unit 140, the content information output from content information creation unit 161 to management server 500-1 via communication network 300.

Storage unit 181 does not store a content information table among pieces of information stored in storage unit 180 shown in FIG. 2 (while storing others including biological information table and threshold value table).

Management server 500-1 stores the content information transmitted from communication terminal 100-2. When content information is requested from communication terminal 200-1, management server 500-1 transmits the stored content information to communication terminal 200-1 via communication network 300.

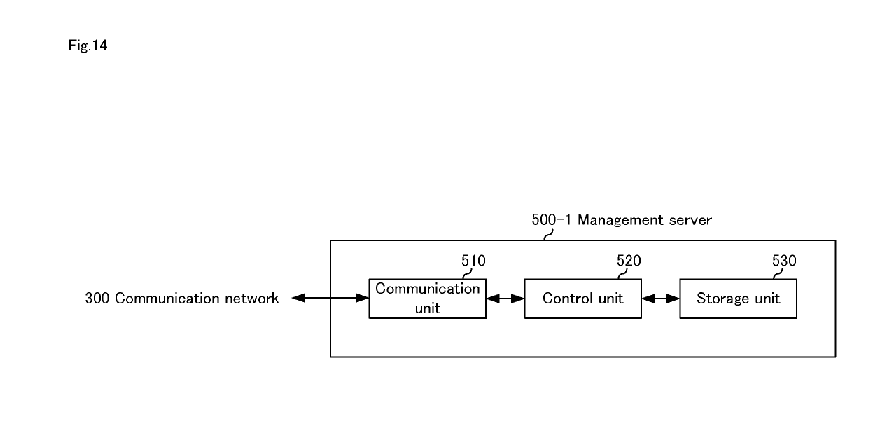

Management server 500-1 shown in FIG. 12 includes, as shown in FIG. 14, communication unit 510, control unit 520, and storage unit 530. FIG. 14 shows an example of the main components, according to the embodiment, from among the components arranged in management server 500-1 shown in FIG. 12.

Communication unit 510 has a communication interface function for performing communication with communication network 300.

Control unit 520 writes and stores the content information transmitted from communication terminal 100-2 in storage unit 530. Control unit 520 provides (transmits) the content information stored in storage unit 530 to communication terminal 200-1 via communication network 300 by using communication unit 510. Accordingly, control unit 520 plays the role of content providing unit 170 according to the first embodiment.

Storage unit 530 stores the content information written by control unit 520. Details on the content information stored in storage unit 530 are similar in content to the content information table stored in storage unit 180 according to the first embodiment. Storage unit 530 stores correspondence between a preset viewer and a preset checker as a user related table.

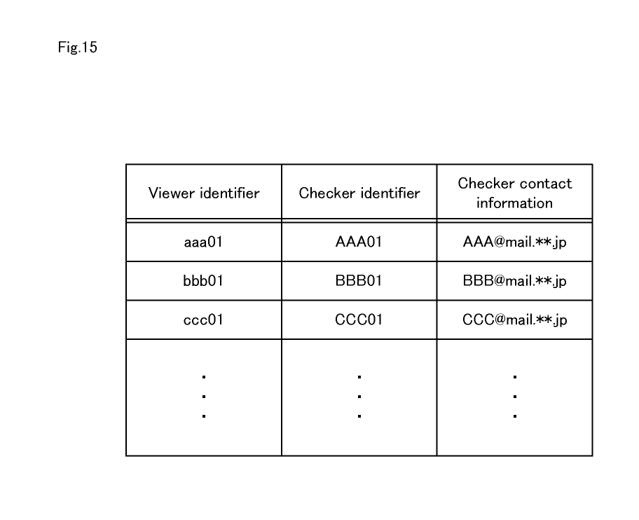

In the user related table stored in storage unit 530 shown in FIG. 14, as shown in FIG. 15, a viewer identifier, a checker identifier, and checker contact information are stored in association. The viewer identifier is identification information capable of identifying a viewer. The checker identifier is identification information capable of identifying a checker. The checker contact information is address information used when the content information is transmitted to the checker. Accordingly, correspondence is set between the viewer and the checker in storage unit 530 and, when the content information is requested from the checker, control unit 520 can identify content information about which viewer will be transmitted to the checker by referring to the correspondence. For example, when content information is requested from the checker of a checker identifier of “AAA01”, control unit 520 recognizes that the content information of a viewer identifier “aaa01” associated with the checker identifier “AAA01” in the user related table stored in storage unit 530 should be transmitted.

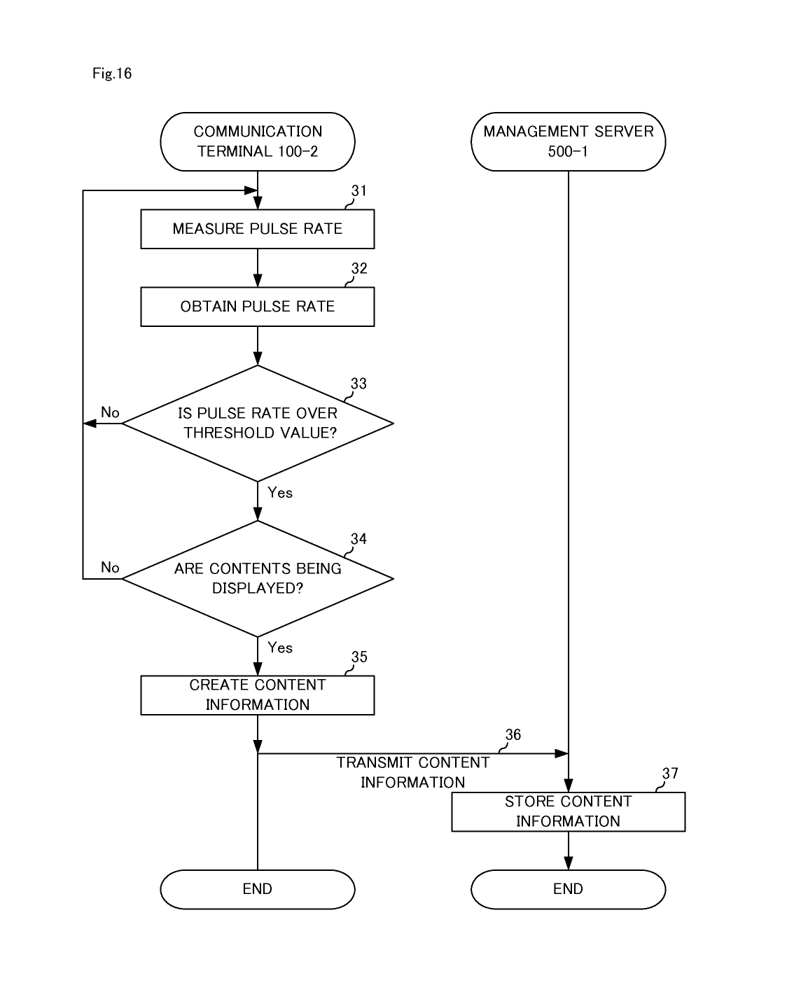

Hereinafter, an information processing method in the embodiment shown in FIG. 12 will be described. First, the processing to create content information at communication terminal 100-2 and management server 500-1 shown in FIG. 12 will be described by referring to FIG. 16. An example where biological information is a pulse rate will be described. An example where output unit 150 outputs content by displaying the content will be described.

First, in step 31, biological information measurement unit 110 attached to the viewer operating communication terminal 100-2 measures the pulse rate of the viewer at a predetermined timing. In step 32, biological information acquisition unit 120 acquires the pulse rate measured by biological information measurement unit 110 from biological information measurement unit 110. Then, biological information acquisition unit 120 writes and stores the acquired pulse rate in the biological information table of storage unit 181.

In step 33, condition determination unit 130 determines whether the pulse rate stored in the biological information table of storage unit 181 exceeds the threshold value preset in the threshold value table of storage unit 181. When condition determination unit 130 determines that the pulse rate stored in the biological information table of storage unit 181 exceeds the threshold value preset in the threshold value table of storage unit 181, in step 34, content information creation unit 161 determines whether output unit 150 is displaying the contents.

When content information creation unit 161 determines that output unit 150 is displaying the contents, in step 35, content information creation unit 160 creates content information indicating the contents that are being displayed to output it to content information providing unit 171. Then, in step 36, content information providing unit 171 transmits the content information output from content creation unit 161 to management server 500-1 via communication network 300 by using communication unit 140. At this time, content information providing unit 171 includes a viewer identifier in the content information to transmit the content information to management server 500-1.

After communication unit 510 of management server 500-1 has received the content information transmitted from communication terminal 100-2, in step 37, control unit 520 writes and stores the content information received by communication unit 510 in storage unit 150.

On the other hand, when condition determination unit 130 determines that the pulse rate stored in the biological information table of storage unit 181 does not exceed the threshold value preset in the threshold value table of storage unit 181 in step 33, or when content information creation unit 161 determines that output unit 150 is not displaying the contents in step 34, the processing of step 31 is executed at the next timing.

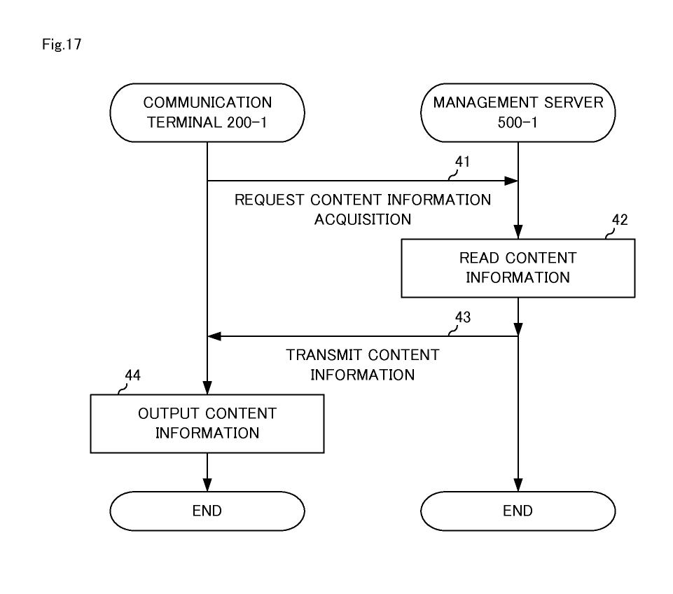

Next, processing when content information is requested from communication terminal 200-1 shown in FIG. 12 will be described referring to FIG. 17.

After input unit 210 of communication terminal 200-1 has received a predetermined input, in step 41, control unit 220 requests acquisition of content information to management server 500-1 via communication network 300 by using communication unit 230. For this request, a request signal enabling recognition of the request to acquire content information at management sever 500-1 can be used. The request signal includes a checker identifier.

After a request has been made to acquire content information from communication terminal 200-1, control unit 520 reads a viewer identifier associated with the checker identifier included in the request signal from the user related table stored in storage unit 530. Then, in step 42, control unit 520 reads the content information associated with the read viewer identifier from storage unit 530. Then, in step 43, control unit 520 transmits the read content information to communication terminal 200-1 via communication network 300 by using communication unit 510.

After communication unit 230 has received the content information transmitted from management sever 500-1, in step 44, control unit 220 outputs information indicated by the content information from output unit 240. This information can be a capture screen when the content information includes the capture screen itself, or a URL when the content information includes the URL of a content distribution site.

After management sever 500-1 has received the content information transmitted from communication terminal 100-2, its message can be notified to communication terminal 200-1. An address used for the notification can be the electronic mail address of the checker contact information stored in the user related table of storage unit 530. The processing of communication terminal 200-1 when the notification is received is similar to that described above referring to the sequential diagram of FIG. 11 (processing of communication terminal 100-1 is processing of management server 500-1).

Others can be similar to those of the first embodiment.

### Third Embodiment

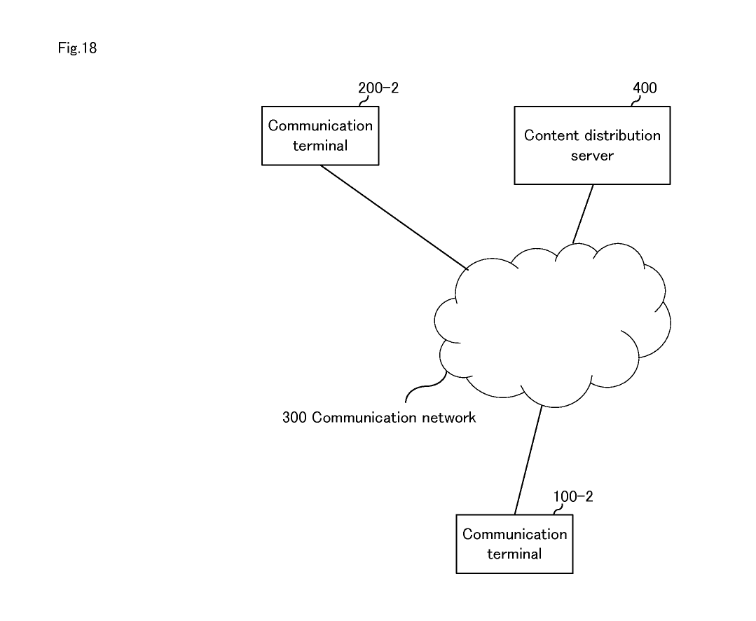

FIG. 18 shows an information processing system according to the third embodiment of the present invention, where communication terminals 100-2 and 200-2 and content distribution server 400 can communicate with one another via communication network 300. In the exemplary embodiment shown in FIG. 18, one communication terminal 100-2, one communication terminal 200-2, and one content distribution server 400 are shown. However, there are no restrictions on the numbers thereof. In the third embodiment, content information created by communication terminal 100-2 is stored by communication terminal 200-2.

Communication terminal 100-2 and content distribution server 400 are similar to those shown in FIG. 12. However, communication terminal 100-2 transmits created content information not to management server 500-1 shown in FIG. 12 but to communication terminal 200-2.

Communication terminal 200-2 is a second information processing terminal operated by a checker.

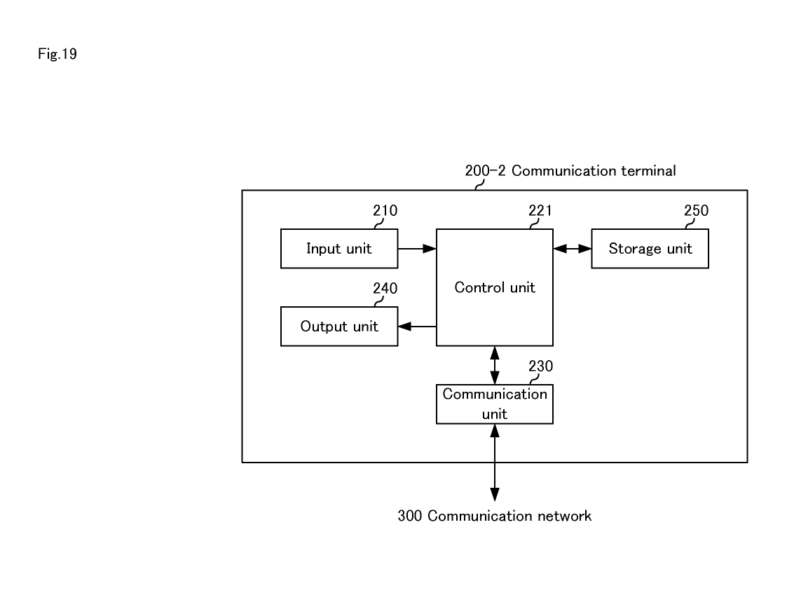

Communication terminal 200-2 shown in FIG. 18 includes, as shown in FIG. 19, input unit 210, control unit 221, communication unit 230, output unit 240, and storage unit 250. FIG. 19 shows an example of main components, according to the embodiment, from among the components arranged in communication terminal 200-2 shown in FIG. 18.

Input unit 210, communication unit 230, and output unit 240 are similar to those shown in FIG. 8.

Control unit 221 has, in addition to the function of control unit 220 shown in FIG. 8, a function for writing and storing the content information transmitted from communication terminal 100-2. After input unit 210 has received a predetermined input by a checker's operation, control unit 221 reads the content information stored in storage unit 250, and outputs information indicated by the read content information from output unit 240.

Storage unit 250 stores the content information written by control unit 221.

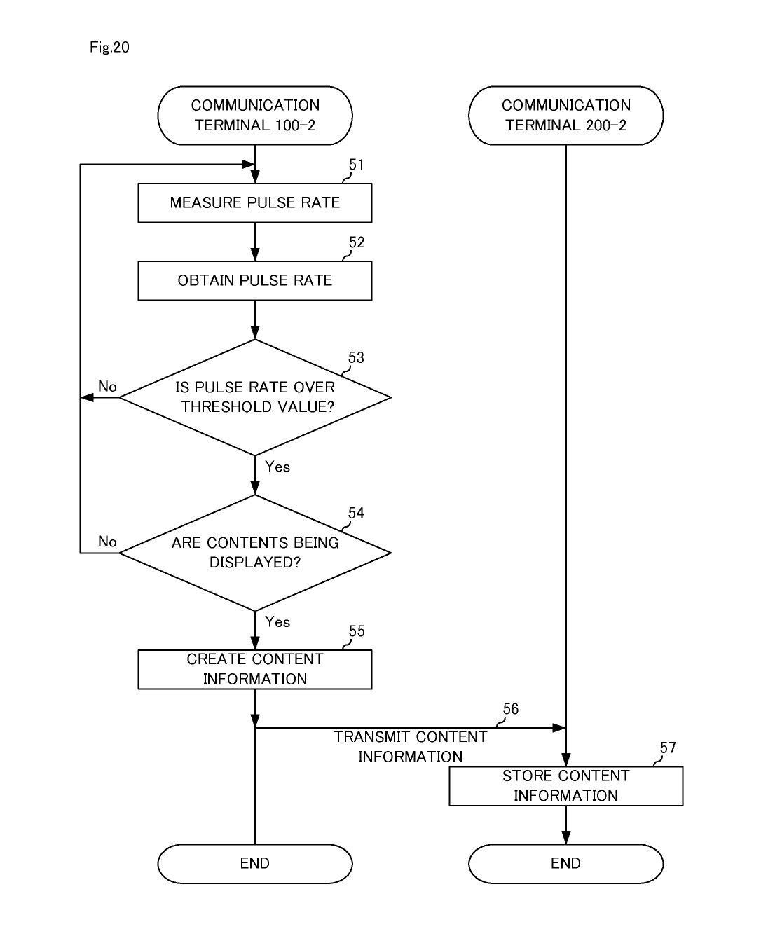

Hereinafter, an information processing method in the embodiment shown in FIG. 18 will be described. First, the processing to create content information at communication terminal 100-2 shown in FIG. 12 will be described by referring to FIG. 20. An example where biological information is a pulse rate will be described. An example where output unit 150 outputs contents by displaying will be described.

First, in step 51, biological information measurement unit 110 attached to a viewer who is operating communication terminal 100-2 measures the pulse rate of the viewer at predetermined timing. In step 52, biological information acquisition unit 120 acquires the pulse rate measured by biological information measurement unit 110 from biological information measurement unit 110. Then, biological information acquisition unit 120 writes and stores the acquired pulse rate in the biological information table of storage unit 181.

In step 53, condition determination unit 130 determines whether the pulse rate stored in the biological information table of storage unit 181 exceeds the threshold value preset in the threshold value table of storage unit 181. When condition determination unit 130 determines that the pulse rate stored in the biological information table of storage unit 181 exceeds the threshold value preset in the threshold value table of storage unit 181, in step 54, content information creation unit 161 determines whether output unit 150 is displaying the contents.

When content information creation unit 161 determines that output unit 150 is displaying the contents, in step 55, content information creation unit 160 creates content information indicating the contents that are being displayed to output it to content information providing unit 171. Then, in step 56, content information providing unit 171 transmits the content information output from content creation unit 161 to communication terminal 200-2 via communication network 300 by using communication unit 140.

After communication unit 230 of communication terminal 200-2 has received the content information transmitted from communication terminal 100-2, in step 57, control unit 221 writes and stores the content information received by communication unit 230 in storage unit 250.

On the other hand, when condition determination unit 130 determines that the pulse rate stored in the biological information table of storage unit 181 does not exceed the threshold value preset in the threshold value table of storage unit 181 in step 53, or when content information creation unit 161 determines that output unit 150 is not displaying the contents in step 54, the processing of step 51 is executed at the next timing.

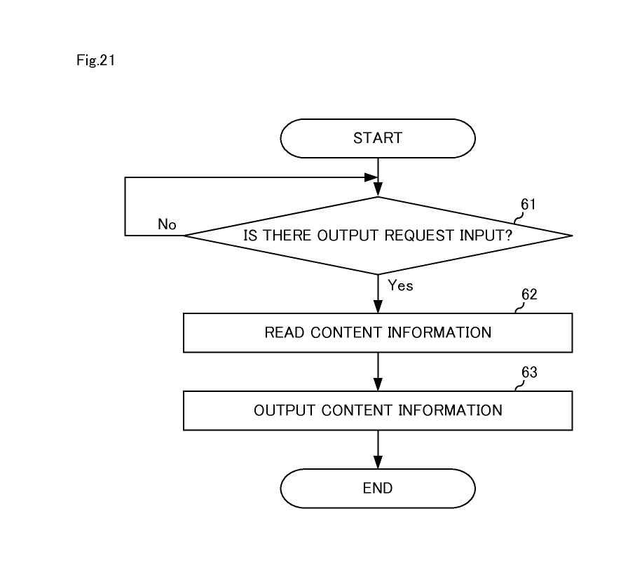

Next, output processing in communication terminal 200-2 shown in FIG. 18 will be described by referring to FIG. 21.

First, in step 61, control unit 221 determines whether outputting of content information has been requested from outside communication terminal 200-2. Control unit 221 executes this determination by determining whether input unit 210 has received a predetermined input for requesting the outputting of the content information. When control unit 221 determines that the outputting of content information has been requested from outside communication terminal 200-2, in step 62, control unit 221 reads content information stored in storage unit 250. Then, in step 63, control unit 221 outputs information indicated by the read content information from output unit 240. This information can be a capture screen when the content information includes the capture screen itself, or a URL when the content information includes the URL of a content distribution site.

### Fourth Embodiment

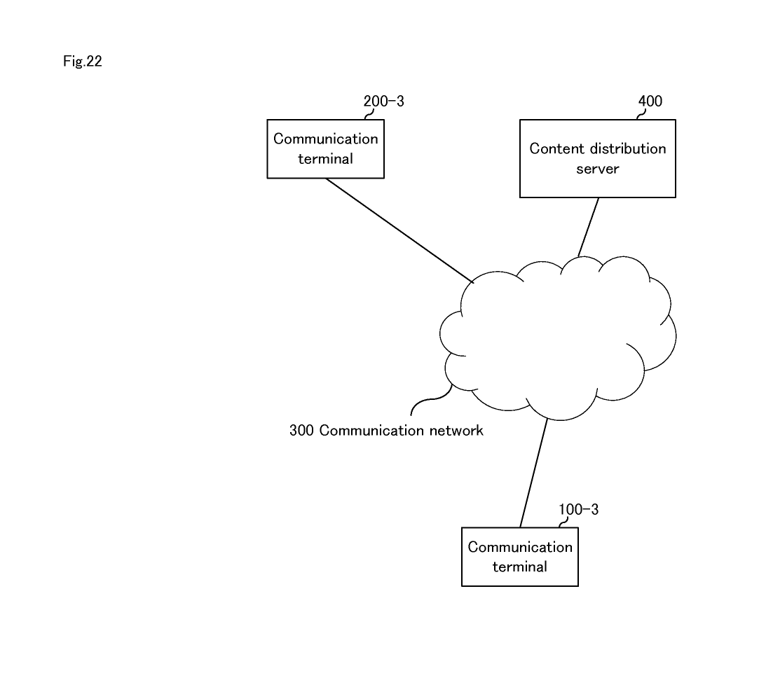

FIG. 22 shows an information processing system according to the fourth embodiment of the present invention, where communication terminals 100-3 and 200-3 and content distribution server 400 can communicate with one another via communication network 300. In the exemplary embodiment shown in FIG. 22, one communication terminal 100-3, one communication terminal 200-3, and one content distribution server 400 are shown. However, there are no restrictions on the number of terminals and servers thereof. In the fourth embodiment, communication terminal 200-3 determines whether measured content information satisfies conditions.

Content distribution server 400 is similar to that shown in FIG. 1.

Communication terminal 100-3 is a first information processing terminal operated by a viewer.

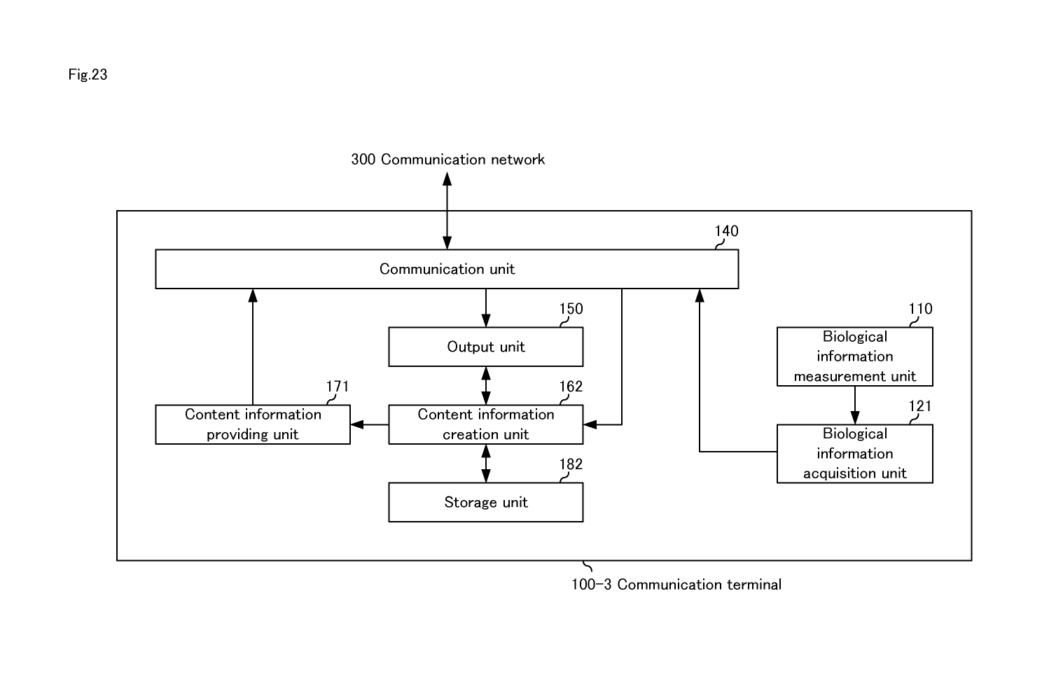

Communication terminal 100-3 shown in FIG. 22 includes, as shown in FIG. 23, biological information measurement unit 110, biological information acquisition unit 121, communication unit 140, output unit 150, content information creation unit 162, content information providing unit 171, and storage unit 182. FIG. 23 shows an example of main components, according to the embodiment, from among the components arranged in communication terminal 100-3 shown in FIG. 22.

Biological information measurement unit 110, communication unit 140, output unit 150 are similar to those shown in FIG. 2. Content information providing unit 171 is similar to that shown in FIG. 13.

Biological information acquisition unit 121 acquires biological information measured by biological information measurement unit 110. Biological information acquisition unit 120 transmits the acquired biological information to communication terminal 200-3 via communication network 300 by using communication unit 140.

Content information creation unit 162 creates content information after the creation of the content information has bas been requested from communication terminal 200-3. A transmission delay (time lag) is generated in communication between the point of time when biological information satisfying predetermined conditions is measured and the point of time when the measured biological information is transmitted to communication terminal 200-3, communication terminal 200-3 determines whether the biological information satisfies the conditions, and the request of the creation of the content information transmitted from communication terminal 200-3 is received. Because of this transmission delay, if content information indicating the contents of information that are output from output unit 150 is created when the creation request of the content information is received, there is a possibility that content information that is different in content from information output from output unit 150 when the measured biological information satisfies the conditions will be created.

Accordingly, biological information acquisition unit 121 also acquires measurement time when it acquires the biological information measured by biological information measurement unit 110, and transmits it to communication terminal 200-3. When it is determined that the biological information satisfies the conditions, communication terminal 200-3 transmits the measurement time received with the biological information to communication terminal 100-3 together with the creation request of content information. Content information creation unit 162 stores, for example, contents output from output unit 150 together with output time in storage unit 182 for several minutes. When it receives the request to create the content information from communication terminal 200-3, content information creation unit 162 reads the contents stored in storage unit 180 together with the output time similar to the measurement time simultaneously received with the request from storage unit 180, and creates content information based on the read contents.

Content information creation unit 162 outputs the created content information to content information providing unit 171.

Storage unit 182 stores the contents output from output unit 150 together with the output time for a predetermined time. However, storage unit 182 stores neither a biological information table nor a threshold value table.

Communication terminal 200-3 is a second information processing terminal operated by a checker.

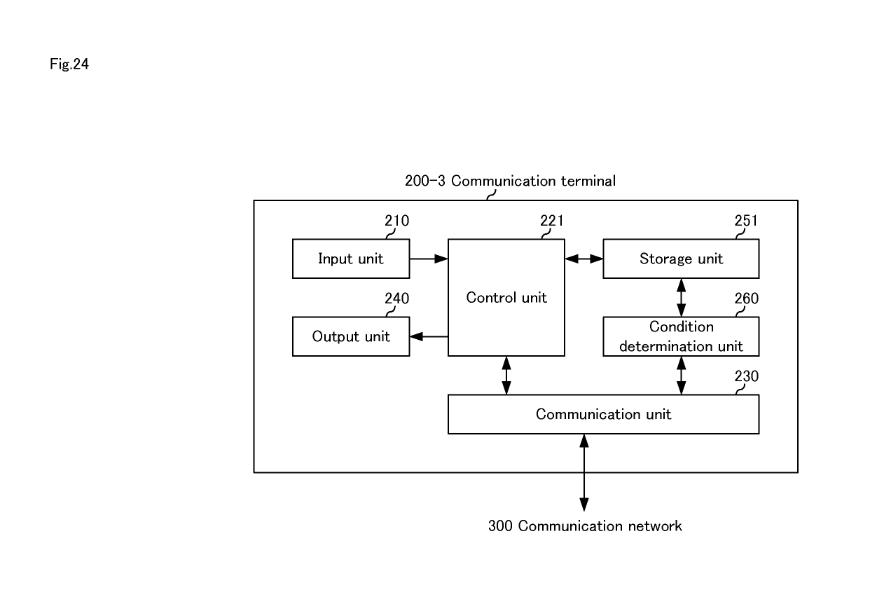

Communication terminal 200-3 shown in FIG. 22 includes, as shown in FIG. 24, input unit 210, control unit 221, communication unit 230, output unit 240, storage unit 251, and condition determination unit 260. FIG. 24 shows an example of main components, according to the embodiment, from among the components arranged in communication terminal 200-3 shown in FIG. 22.

Input unit 210, communication unit 230, and output unit 240 are similar to those shown in FIG. 8. Control unit 221 is similar to that shown in FIG. 19.

Storage unit 251 stores a threshold value used as a reference by condition determination unit 260 beforehand. The storage form of the threshold value can be similar to the threshold value table form shown in FIG. 4 or FIG. 6. Storage unit 251 stores the content information written by control unit 221.

Condition determination unit 260 determines whether the biological information transmitted from communication terminal 100-3 satisfies conditions stored in storage unit 251. When the biological information transmitted from communication terminal 100-3 exceeds the threshold value stored in storage unit 251, condition determination unit 260 determines that the biological information satisfies the conditions. When a state where the biological information transmitted from communication terminal 100-3 exceeds the threshold value stored in storage unit 251 continues for a predetermined time, condition determination unit 260 can determine that the biological information satisfies the conditions. After determinating that the biological information satisfies the conditions, condition determination unit 260 requests the creation of content information to communication terminal 100-3 via communication network 300 by using communication unit 230. For this request, a request signal enabling recognition of the request to create the content information at communication terminal 100-3 can be used. The request signal includes the measurement time received together with the biological information.

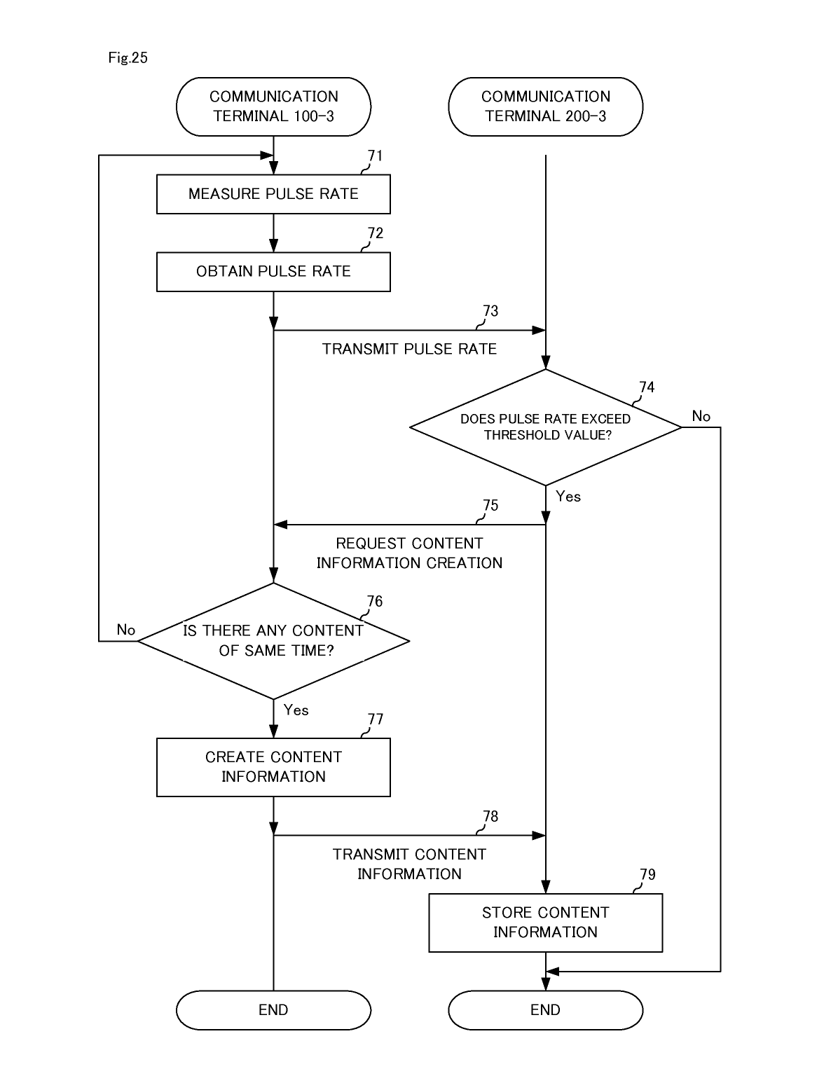

Hereinafter, an information processing method in the embodiment shown in FIG. 22 will be described. First, the processing to create content information at communication terminals 100-3 and 20-3 shown in FIG. 22 will be described referring to FIG. 25. An example where biological information is a pulse rate will be described. An example where output unit 150 outputs contents by displaying will be described.

First, content information creation unit 162 writes contents that are being displayed by output unit 150 in storage unit 182 in association with the output time. For the writing timing, a predetermined period or time can be used. Storage unit 182 deletes the contents after a predetermined time has elapsed from the writing of the contents. The contents written in storage unit 182 by content information creation unit 162 indicate the contents that are being displayed by output unit 150, or the contents themselves. Any information can be used as long as it enables creation of content information.

In step 71, biological information measurement unit 110 attached to a viewer who is operating communication terminal 100-3 measures the pulse rate of the viewer at a predetermined timing. In step 72, biological information acquisition unit 121 acquires the pulse rate measured by biological information measurement unit 110 from biological information measurement unit 110. At this time, biological information acquisition unit 121 also acquires the measurement time of the pulse rate by biological information measurement unit 110. Then, in step 73, biological information acquisition unit 121 transmits the acquired pulse rate to communication terminal 200-3 via communication network 300 by using communication unit 140. At this time, biological information acquisition unit 121 also transmits the measurement time to communication terminal 200-3 together with the acquired pulse rate.

After communication unit 230 of communication terminal 200-3 has received the pulse rate and the measurement time transmitted from communication terminal 100-3, in step 74, condition determination unit 260 determines whether the pulse rate received by communication unit 230 exceeds a threshold value preset in the threshold value table of storage unit 251. When condition determination unit 260 determines that the pulse rate received by communication unit 230 exceeds the threshold value preset in the threshold value table of storage unit 251, in step 75, condition determination unit 260 requests the creation of content information to communication terminal 100-3 via communication network 300 by using communication unit 230. At this time, condition determination unit 260 also transmits the measurement time transmitted from transmission terminal 100-3 to communication terminal 100-3. On the other hand, when condition determination unit 260 determines that the pulse rate received by communication unit 230 does not exceed the threshold value preset in the threshold value table of storage unit 251, the processing is ended.

Then, after communication unit 140 of communication terminal 100-3 has received the request to create the content information from communication terminal 200-3, in step 76, content information creation unit 162 determines whether the contents of the output time similar to the measurement time received together with the request has been stored in storage unit 182.

When content information creation unit 162 determines that the contents of the output time same as the measurement time have been stored in storage unit 182, content information creation unit 162 reads the contents from storage unit 182. Then, in step 77, content information creation unit 162 creates content information based on the read contents to output it to content information providing unit 171. In step 78, content information providing unit 171 transmits the content information output from content information creation unit 162 to communication terminal 200-3 via communication network 300 by using communication unit 140.

After communication unit 230 of communication terminal 200-3 has received the content information transmitted from communication terminal 100-3, in step 79, control unit 221 writes and stores the content information received by communication unit 230 in storage unit 251.

On the other hand, when content information creation unit 162 determines that the contents of the output time similar to the measurement time has not been stored in storage unit 182 in step 76, the processing of step 71 is executed at the next timing.

Depending on the measurement timing of the biological information and the output timing of the contents, the measurement time and the output time do not necessarily match each other. Accordingly, by securing a certain margin, content information creation unit 162 can search for the contents of the output time included in several seconds or several minutes before or after the measurement time transmitted from communication terminal 200-3 in storage unit 182.

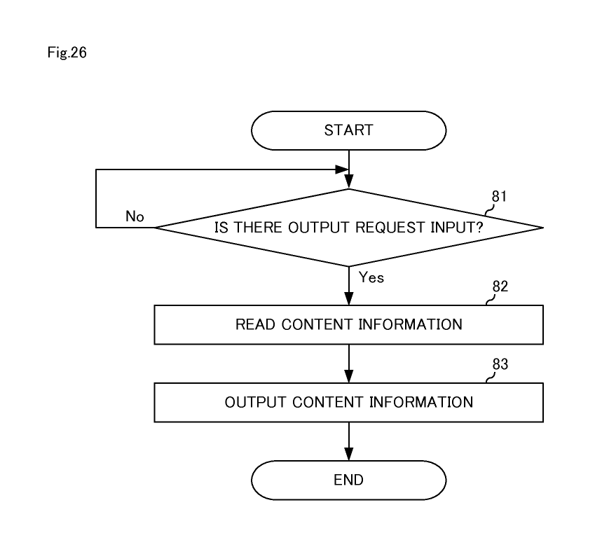

Next, the processing to output content information at communication terminal 200-3 shown in FIG. 22 will be described referring to FIG. 26.

First, in step 81, control unit 221 determines whether outputting of content information has been requested from outside communication terminal 200-3. Control unit 221 executes this determination by determining whether input unit 210 has received a predetermined input for requesting outputting of the content information. When control unit 221 determines that outputting of content information has been requested from outside communication terminal 200-2, in step 82, control unit 221 reads content information stored in storage unit 251. Then, in step 83, control unit 221 outputs information indicated by the read content information from output unit 240. This information can be a capture screen when the content information includes the capture screen itself, or a URL when the content information includes the URL of a content distribution site.

As in the case of the second embodiment, communication terminal 100-3 can transmit the created content information to management server 500-1, and management server 500-1 can store the content information. In this case, after the contents information has been received from communication terminal 100-3, management server 500-1 can store the content information, and read the content information when the content information is requested from communication terminal 200-3 to transmit it to communication terminal 200-3. After the content information has been received from communication terminal 100-3, management server 500-1 can notify its message to communication terminal 200-3.

Other processes can be similar to those of the first embodiment.

### Fifth Embodiment

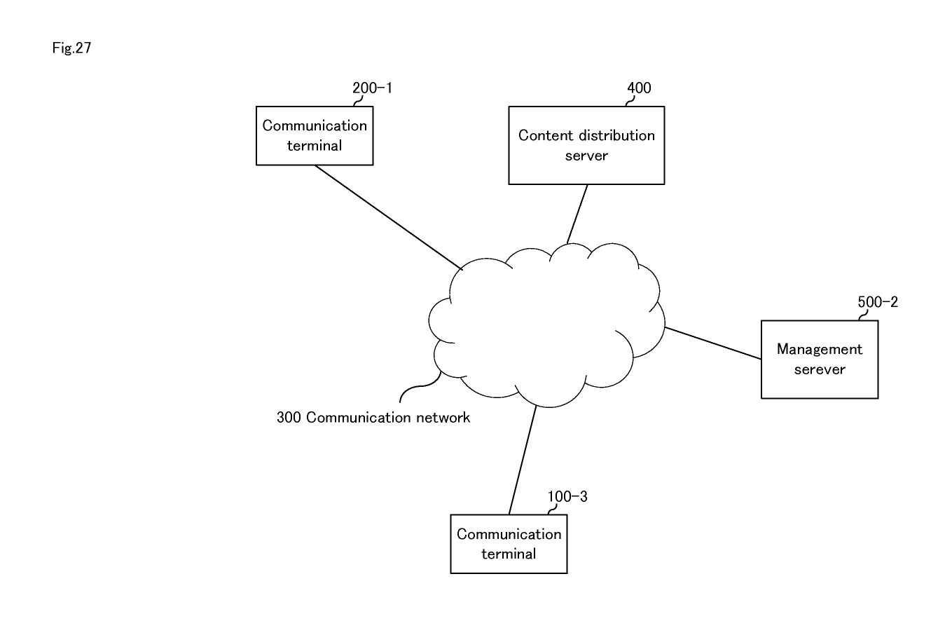

FIG. 27 shows an information processing system according to the fifth embodiment of the present invention, where communication terminals 100-3 and 200-1, content distribution server 400, and management server 500-2 can communicate with one another via communication network 300. In the exemplary embodiment shown in FIG. 27, one communication terminal 100-3, one communication terminal 200-1, and one content distribution server 400 are shown. However, there are no restrictions on the numbers thereof. In the fifth embodiment, management server 500-2 determines whether measured biological information satisfies conditions.

Communication terminal 200-1 and content distribution server 400 are similar to those shown in FIG. 1. Communication terminal 100-3 is similar to that shown in FIG. 22. However, biological information acquisition unit 121 of communication terminal 100-3 transmits biological information acquired from biological information measurement unit 110 to management server 500-2 via communication network 300 by using communication terminal 140. Content information creation unit 162 of communication terminal 100-3 creates content information after the creation of content information has been requested from management server 500-2. Content information providing unit 172 transmits the content information to management server 500-2 via communication network 300 by using communication terminal 140.

Management server 500-2 determines whether biological information transmitted from communication terminal 100-3 satisfies conditions, and requests the creation of content information to communication terminal 100-3 when the biological information satisfies the conditions. Management server 500-2 stores the content information transmitted from communication terminal 100-3. When content information is requested from communication terminal 200-1, management server 500-2 transmits the stored content information to communication terminal 200-1 via communication network 300.

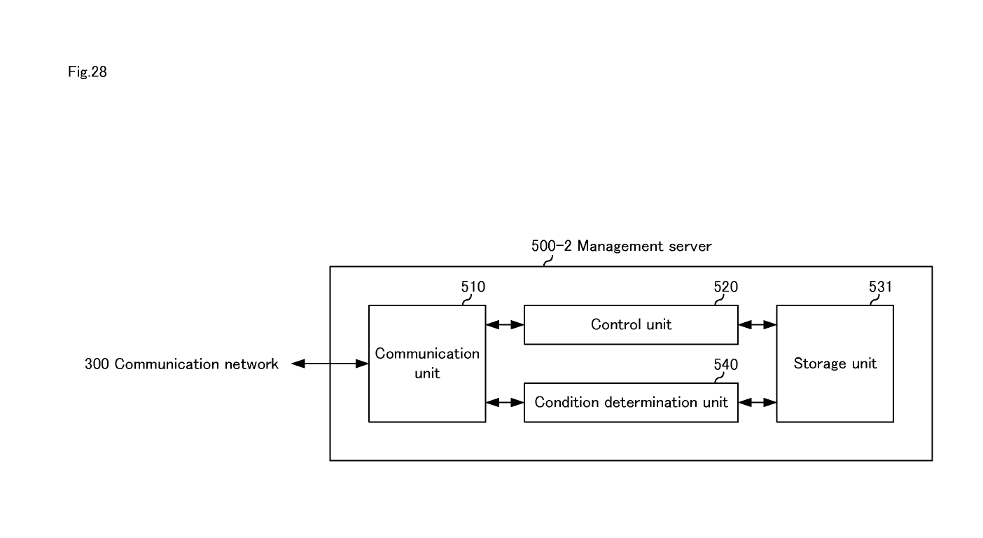

Management server 500-2 shown in FIG. 27 includes, as shown in FIG. 28, communication unit 510, control unit 520, storage unit 530, and condition determination unit 540. FIG. 28 shows an example of the main components, according to the embodiment, from among the components arranged in management server 500-2 shown in FIG. 27.

Communication unit 510 and control unit 520 are similar to those shown in FIG. 14.

Storage unit 531 stores content information written by control unit 520. Details on the content information stored in storage unit 531 are similar in content to the content information table stored in storage unit 180 according to the first embodiment. Storage unit 531 stores correspondence between a preset viewer and a preset checker as a user related table. The user related table is similar to that shown in FIG. 15. Storage unit 531 stores a threshold value used as a reference for making a determination by condition determination unit 540 beforehand. The storage form of the threshold value can be similar to the threshold value table shown in FIG. 4 or FIG. 6.

Condition determination unit 540 determines whether the biological information transmitted from communication terminal 100-3 satisfies the conditions stored in storage unit 531. When the biological information transmitted from communication terminal 100-3 satisfies the threshold value stored in storage unit 531, condition determination unit 130 determines that the biological information satisfies the conditions. Condition determination unit 130 can determine that the biological information satisfies the conditions when a state where the biological information transmitted from communication terminal 100-3 satisfies the threshold value stored in storage unit 531 continues for a predetermined time. When it is determined that the biological information satisfies the conditions, condition determination unit 540 requests the creation of content information to communication terminal 100-3 via communication network 300 by using communication unit 510. For this request, a request signal enabling recognition of the request to acquire of the content information at communication terminal 100-3 can be used. The request signal includes measurement time received together with the biological information.

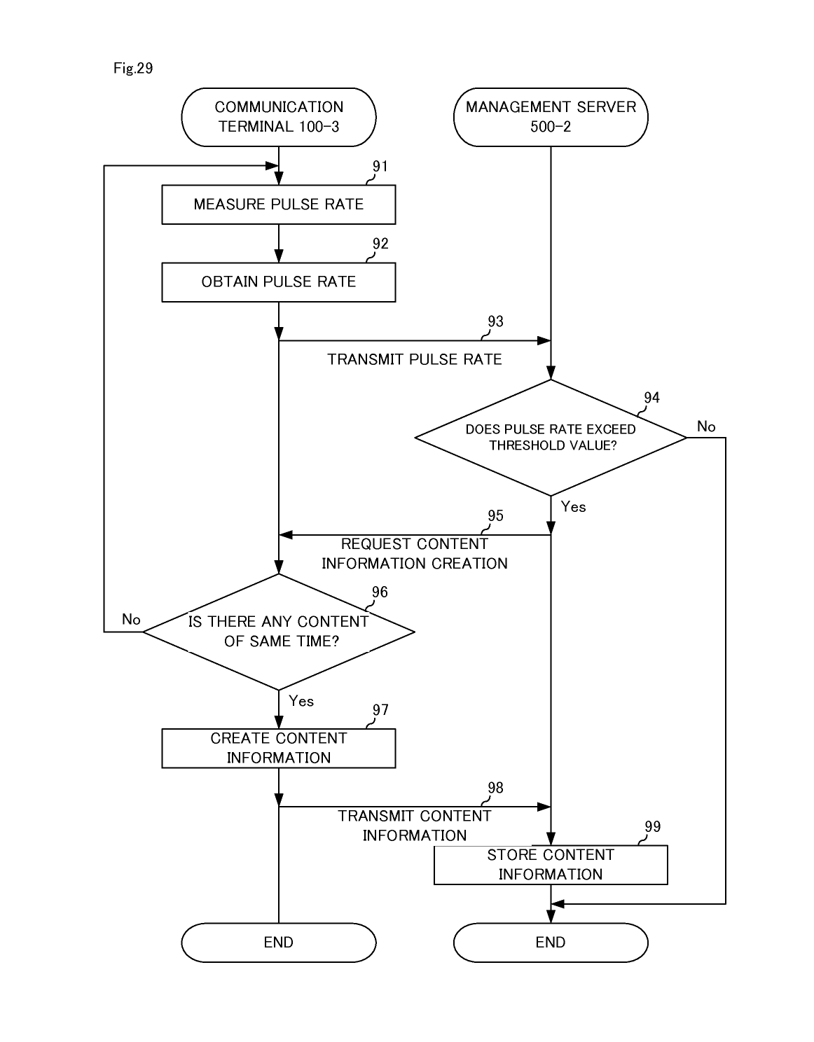

Hereinafter, an information processing method in the embodiment shown in FIG. 27 will be described. First, the processing to create content information at communication terminal 100-3 and management server 500-2 shown in FIG. 27 will be described referring to FIG. 29. An example where biological information is a pulse rate will be described. An example where output unit 150 outputs contents by displaying will be described.

First, content information creation unit 162 writes contents that are being displayed by output unit 150 in storage unit 182 in association with the output time. For the writing timing, a predetermined period or time can be used. Storage unit 182 deletes the contents after a predetermined time has elapsed from the writing of the contents. The contents written in storage unit 182 by content information creation unit 162 indicate the contents that are being displayed by output unit 150, or the contents themselves. Any information can be used as long as it enables creation of content information.

In step 91, biological information measurement unit 110 attached to a viewer who is operating communication terminal 100-3 measures the pulse rate of the viewer at a predetermined timing. In step 92, biological information acquisition unit 121 acquires the pulse rate measured by biological information measurement unit 110 from biological information measurement unit 110. At this time, biological information acquisition unit 121 also acquires the measurement time of the pulse rate by biological information measurement unit 110. Then, in step 93, biological information acquisition unit 121 transmits the acquired pulse rate to management server 500-2 via communication network 300 by using communication unit 140. At this time, biological information acquisition unit 121 also transmits the measurement time to management server 500-2 together with the acquired pulse rate.

After communication unit 510 of management server 500-2 has received the pulse rate and the measurement time transmitted from communication terminal 100-3, in step 94, condition determination unit 540 determines whether the pulse rate received by communication unit 510 exceeds a threshold value preset in the threshold value table of storage unit 531. When condition determination unit 540 determines that the pulse rate received by communication unit 510 exceeds the threshold value preset in the threshold value table of storage unit 531, in step 95, condition determination unit 540 requests the creation of content information to communication terminal 100-3 via communication network 300 by using communication unit 510. At this time, condition determination unit 540 also transmits the measurement time transmitted from transmission terminal 100-3 to communication terminal 100-3. On the other hand, when condition determination unit 540 determines that the pulse rate received by communication unit 510 does not exceed the threshold value preset in the threshold value table of storage unit 531, the processing is ended.

Then, after communication unit 140 of communication terminal 100-3 has received the request to create the content information from management server 500-2, in step 96, content information creation unit 162 determines whether the contents of the output time similar to the measurement time received together with the request have been stored in storage unit 182.

When content information creation unit 162 determines that the contents of the output time similar to the measurement time have been stored in storage unit 182, content information creation unit 162 reads the contents from storage unit 182. Then, in step 97, content information creation unit 162 creates content information based on the read contents to output it to content information providing unit 171. In step 98, content information providing unit 171 transmits the content information output from content information creation unit 162 to management server 500-2 via communication network 300 by using communication unit 140.

After communication unit 510 of management server 500-2 has received the content information transmitted from communication terminal 100-3, in step 99, control unit 520 writes and stores the content information received by communication unit 510 in storage unit 531. After the content information has been received from communication terminal 100-3, management server 500-2 can notify its message to communication terminal 200-3 by using a checker identifier (checker contact information) associated with a viewer identifier included in the content information.

On the other hand, when content information creation unit 162 determines that the contents of the output time similar to the measurement time have not been stored in storage unit 182 in step 96, the processing of step 91 is executed at next timing.

Depending on the measurement timing of the biological information and the output timing of the contents, the measurement time and the output time do not necessarily match each other. Accordingly, by securing a certain margin, content information creation unit 162 can search for the content of the output time that is included in a period of several seconds or several minutes before or after the measurement time transmitted from communication terminal 200-3 in storage unit 182.

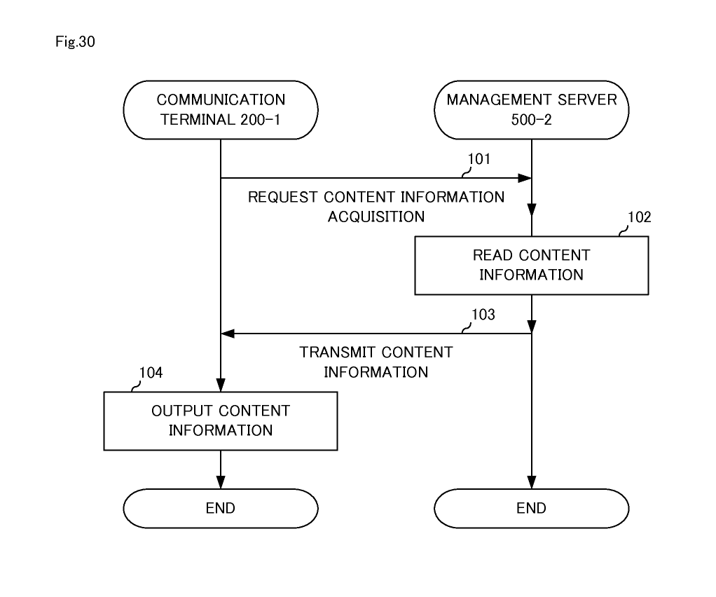

Next, the output processing of content information in management server 500-2 and communication terminal 200-1 shown in FIG. 27 will be described referring to FIG. 30.

After input unit 210 of communication terminal 200-1 has received a predetermined input, in step 101, control unit 220 requests acquisition of content information to management server 500-2 via communication network 300 by using communication unit 230. For this request, a request signal enabling recognition of the request to acquire the content information at management server 500-2 is used. The request signal includes a checker identifier.

After a request has been made to acquire content information from communication terminal 200-1, control unit 520 reads a viewer identifier associated with the checker identifier included in the request signal from the user related table stored in storage unit 531. Then, in step 102, control unit 520 reads the content information associated with the read viewer identifier from storage unit 531. Then, in step 103, control unit 520 transmits the read content information to communication terminal 200-1 via communication network 300 by using communication unit 510.

After communication unit 230 has received the content information transmitted from management server 500-2, in step 104, control unit 220 outputs information indicated by the content information from output unit 240. This information can be a capture screen when the content information includes the capture screen itself, or a URL when the content information includes the URL of a content distribution site.

Other processes can be similar to those of the first embodiment.

Hereinafter, an embodiment where biological information measurement unit 110 according to the first to fifth embodiments is disposed physically separately from the communication terminal of the viewer will be described.

### Sixth Embodiment

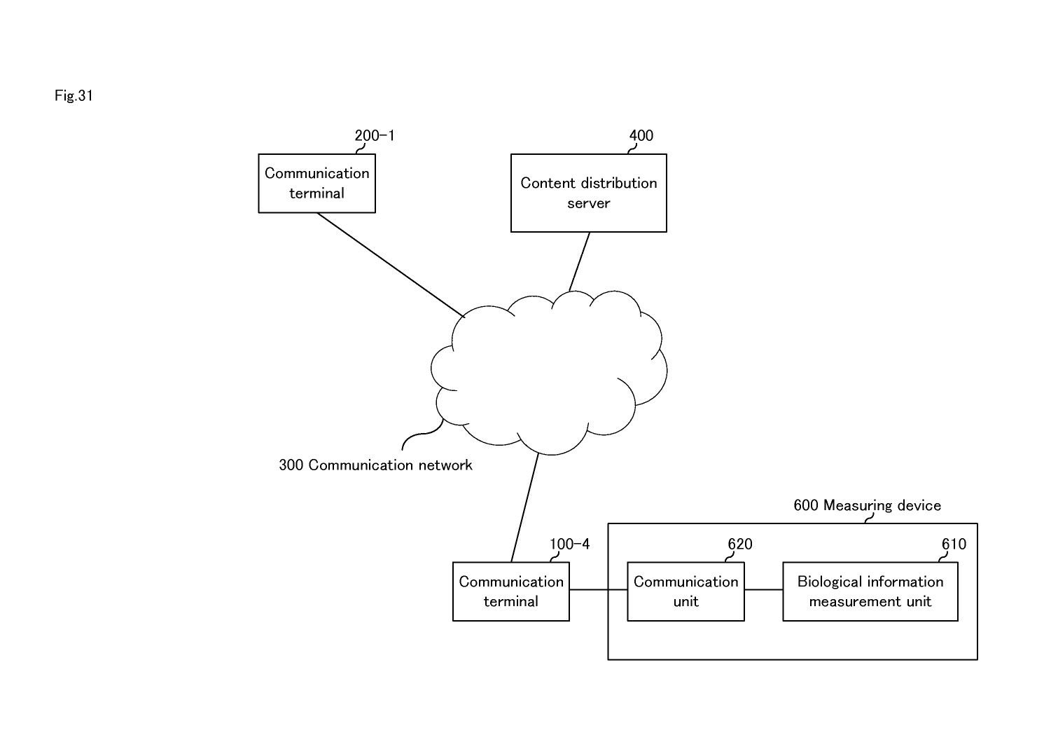

FIG. 31 shows an information processing system where biological information measurement unit 110 disposed in communication terminal 100-1 according to the first embodiment is disposed in measuring device 600 separated as biological information measurement unit 610 into communication unit 620 and communication terminal 100-4 according to the sixth embodiment of the present invention.

Communication terminal 200-1 and content distribution server 400 are similar to those shown in FIG. 1.

Measuring device 600, which has a structure that is attachable to a viewer, transmits the biological information of the viewer measured by biological information measurement unit 610 to communication terminal 100-4 by using communication unit 620. For example, measuring device 600 has a seal structure that is attachable to the vicinity of a breast or a structure such as a thermometer type, a wristband type, or a wristwatch type. When the shape of communication terminal 100-4 is a wristwatch type or a wristband type, measuring device 600 can be disposed on the side of the attachable part of the belt or the wristband that is next to the skin fixed to the casing of communication terminal 100-4. A communication method between communication unit 620 and communication terminal 100-4 is not limited to, for example, wireless communication such as short-distance wireless communication, but can be wired communication.

Communication terminal 100-4 is configured by removing biological information measurement unit 110 from the components of communication terminal 100-1 according to the first embodiment. Biological information acquisition unit 120 of communication terminal 100-4 acquires biological information transmitted from measuring device 600. Communication terminal 100-4 includes a communication unit that acquires (receives) the biological information transmitted from measuring device 600. Processing after communication terminal 100-4 has acquired the biological information is similar to that according to the first embodiment.

### Seventh Embodiment

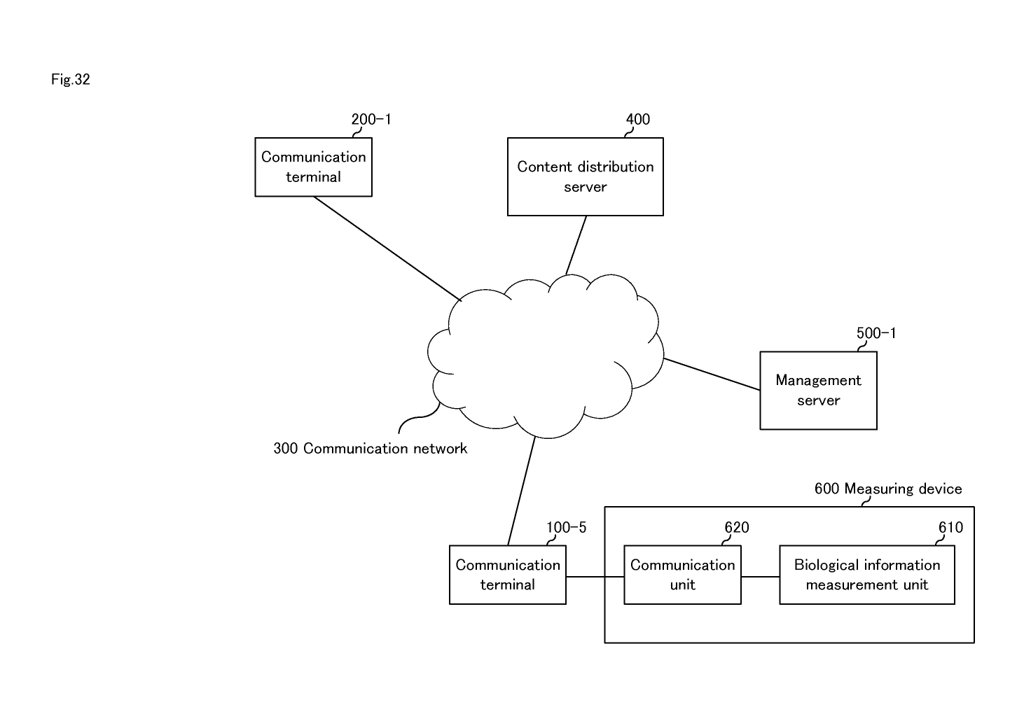

FIG. 32 shows an information processing system where biological information measurement unit 110 disposed in communication terminal 100-2 according to the second embodiment is disposed in measuring device 600 separated as biological information measurement unit 610 into communication unit 620 and communication terminal 100-5 according to the seventh embodiment of the present invention.

Communication terminal 200-1 and content distribution server 400 are similar to those shown in FIG. 12.

Measuring device 600 is similar to that shown in FIG. 31. A communication method between communication unit 620 and communication terminal 100-5 is not limited to, for example, wireless communication such as short-distance wireless communication, but can be wired communication.

Communication terminal 100-5 is configured by removing biological information measurement unit 110 from the components of communication terminal 100-2 according to the second embodiment. Biological information acquisition unit 120 of communication terminal 100-5 acquires biological information transmitted from measuring device 600. Communication terminal 100-5 includes a communication unit that acquires (receives) the biological information transmitted from measuring device 600. Processing after communication terminal 100-5 has acquired the biological information is similar to that according to the second embodiment.

### Eighth Embodiment

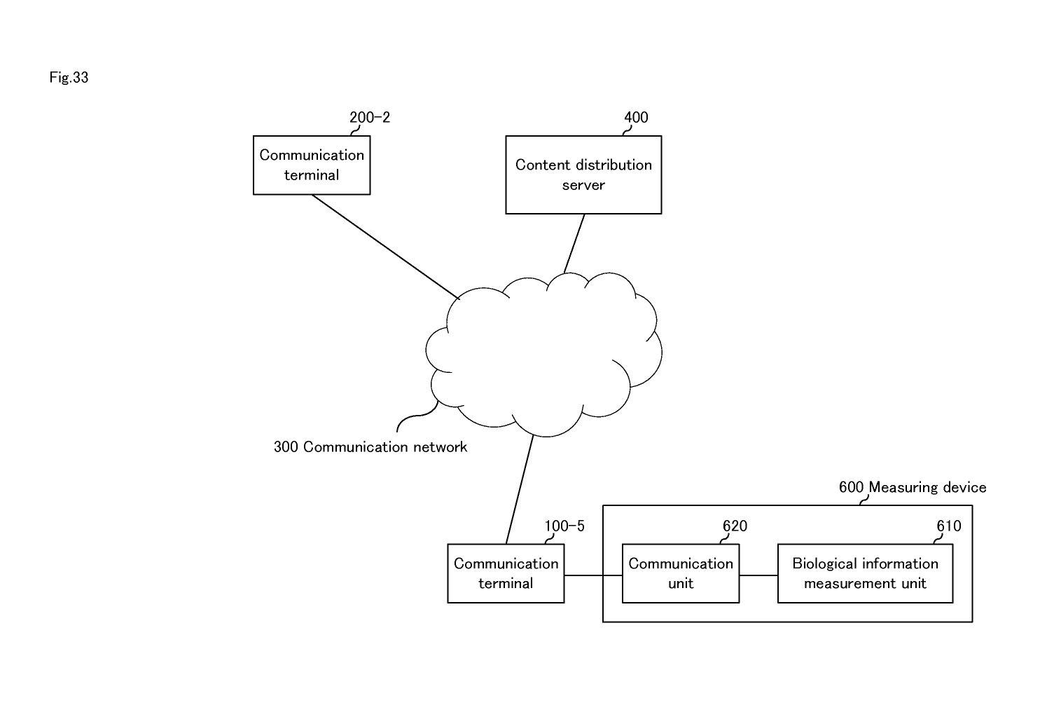

FIG. 33 shows an information processing system where biological information measurement unit 110 disposed in communication terminal 100-2 according to the third embodiment is disposed in measuring device 600 separated as biological information measurement unit 610 into communication unit 620 and communication terminal 100-5 according to the eighth embodiment of the present invention.

Communication terminal 200-2 and content distribution server 400 are similar to those shown in FIG. 18.

Measuring device 600 is similar to that shown in FIG. 32.

Communication terminal 100-5 is configured by removing biological information measurement unit 110 from the components of communication terminal 100-2 according to the third embodiment. Biological information acquisition unit 120 of communication terminal 100-5 acquires biological information transmitted from measuring device 600. Communication terminal 100-5 includes a communication unit that acquires (receives) the biological information transmitted from measuring device 600. Processing after communication terminal 100-5 has acquired the biological information is similar to that according to the third embodiment.

### Ninth Embodiment

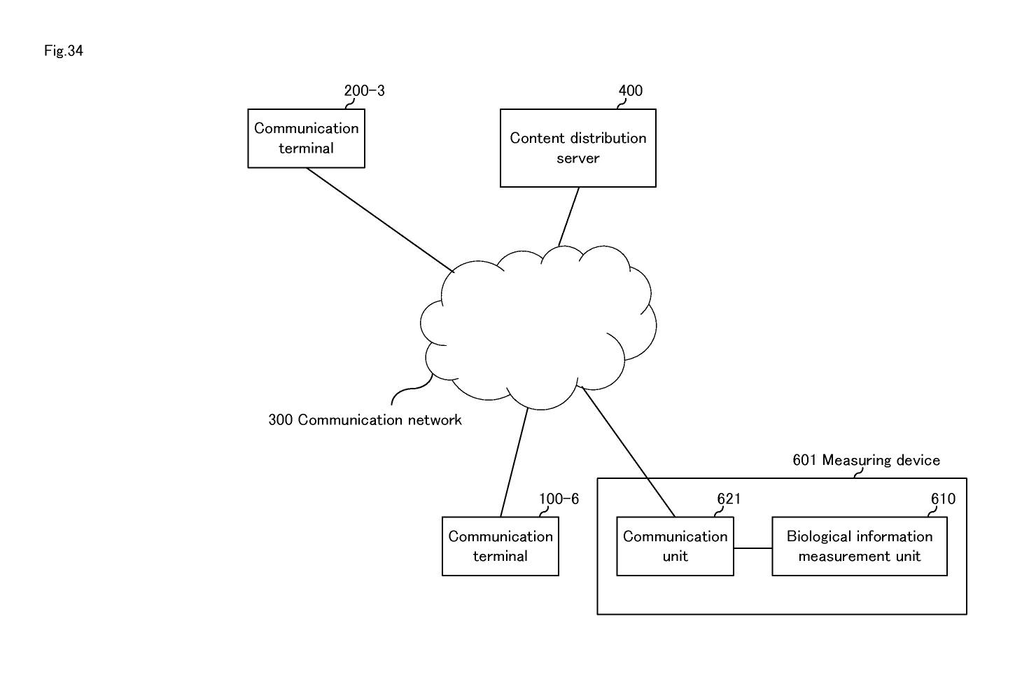

FIG. 34 shows an information processing system where biological information measurement unit 110 disposed in communication terminal 100-3 according to the fourth embodiment is disposed in measuring device 601 separated as biological information measurement unit 610 into communication unit 621 and communication terminal 100-6 according to the ninth embodiment of the present invention.

Communication terminal 200-3 and content distribution server 400 are similar to those shown in FIG. 22.

Measuring device 601, which has a structure that is attachable to a viewer, transmits the biological information of the viewer measured by biological information measurement unit 610 to communication terminal 200-3 via communication network 300 by using communication unit 621. For example, measuring device 600 has a seal structure that is attachable to the vicinity of a breast or a structure such as a thermometer type, a wristband type, or a wristwatch type. When communication unit 621 transmits the biological information to communication network 300, the biological information can be transmitted via communication unit 140 of communication terminal 100-6.

Communication terminal 100-6 is configured by removing biological information measurement unit 110 and biological information acquisition unit 121 from the components of communication terminal 100-3 according to the fourth embodiment.

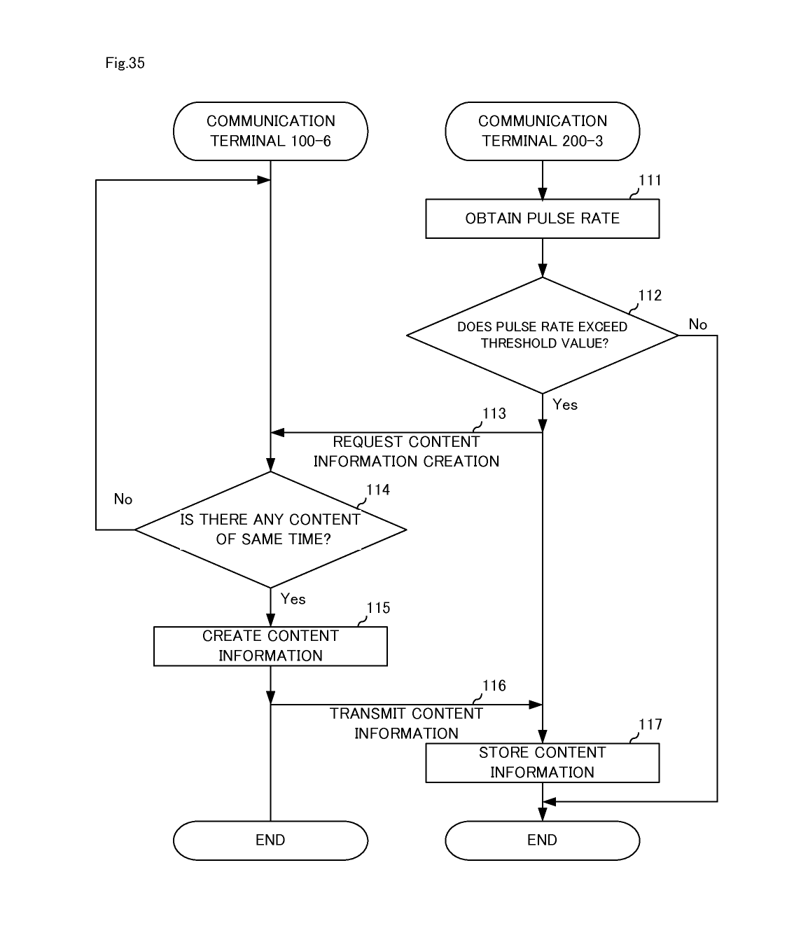

Hereinafter, an information processing method in the embodiment shown in FIG. 34 will be described. First, the processing to create content information at communication terminals 100-6 and 200-3 shown in FIG. 34 will be described referring to FIG. 35. An example where biological information is a pulse rate will be described. An example where output unit 150 outputs content, by displaying the content will be described.

First, content information creation unit 162 writes contents that are being displayed by output unit 150 in storage unit 182 in association with the output time. For the writing timing, a predetermined period or time can be used. Storage unit 182 deletes the contents after a predetermined time has elapsed from the writing of the contents. The contents written in storage unit 182 by content information creation unit 162 indicate the contents that are being displayed by output unit 150, or the contents themselves. Any information can be used as long as it enables creation of content information.

Biological information measurement unit 610 of measuring device 601 attached to a viewer who is operating communication terminal 100-6 measures the pulse rate of the viewer at a predetermined timing. Then, biological information measurement unit 610 transmits the measured pulse rate together with the measurement time of the pulse rate to communication terminal 200-3 via communication network 300 by using communication unit 621. After communication unit 230 of communication terminal 200-3 has received the pulse rate transmitted from measuring device 601, in step 111, condition determination unit 260 acquires the pulse rate received by communication unit 230. At this time, condition determination unit 260 also acquires the measurement time received simultaneously with the pulse rate.

In step 112, condition determination unit 260 determines whether the acquired pulse rate exceeds a threshold value preset in the threshold value table of storage unit 251. When condition determination unit 260 determines that the acquired pulse rate exceeds the threshold value preset in the threshold value table of storage unit 251, in step 113, condition determination unit 260 requests the creation of content information to communication terminal 100-6 via communication network 300 by using communication unit 230. At this time, condition determination unit 260 also transmits the measurement time received together with the pulse rate to communication terminal 100-3. On the other hand, when condition determination unit 260 determines that the pulse rate received by communication unit 230 does not exceed the threshold value preset in the threshold value table of storage unit 251, the processing is ended.

Then, after communication unit 140 of communication terminal 100-6 has received the request to create content information from communication terminal 200-3, in step 114, content information creation unit 162 determines whether the contents of the output time similar to the measurement time received together with the request have been stored in storage unit 182.

When content information creation unit 162 determines that the contents of the output time similar to the measurement time have been stored in storage unit 182, content information creation unit 162 reads the contents from storage unit 182. Then, in step 115, content information creation unit 162 creates content information based on the read contents to output the content information to content information providing unit 171. In step 116, content information providing unit 171 transmits the content information output from content information creation unit 162 to communication terminal 200-3 via communication network 300 by using communication unit 140.

After communication unit 230 of communication terminal 200-3 has received the content information transmitted from communication terminal 100-6, in step 117, control unit 221 writes and stores the content information received by communication unit 230 in storage unit 251.

On the other hand, when content information creation unit 162 determines that the contents of the output time similar to the measurement time have not been stored in storage unit 182 in step 114, content information creation unit 162 waits for transmission of the request to create content information from communication terminal 200-3.

Depending on the measurement timing of the biological information and the output timing of the contents, the measurement time and the output time do not necessarily match each other. Accordingly, by securing a certain margin, content information creation unit 162 can search for the content of the output time that is included in a period of several seconds or several minutes before or after the measurement time transmitted from communication terminal 200-3 in storage unit 182.

Processing for outputting the content information after the content information has been stored in storage unit 251 of communication terminal 200-3 is similar to that of the fourth embodiment.

### Tenth Embodiment

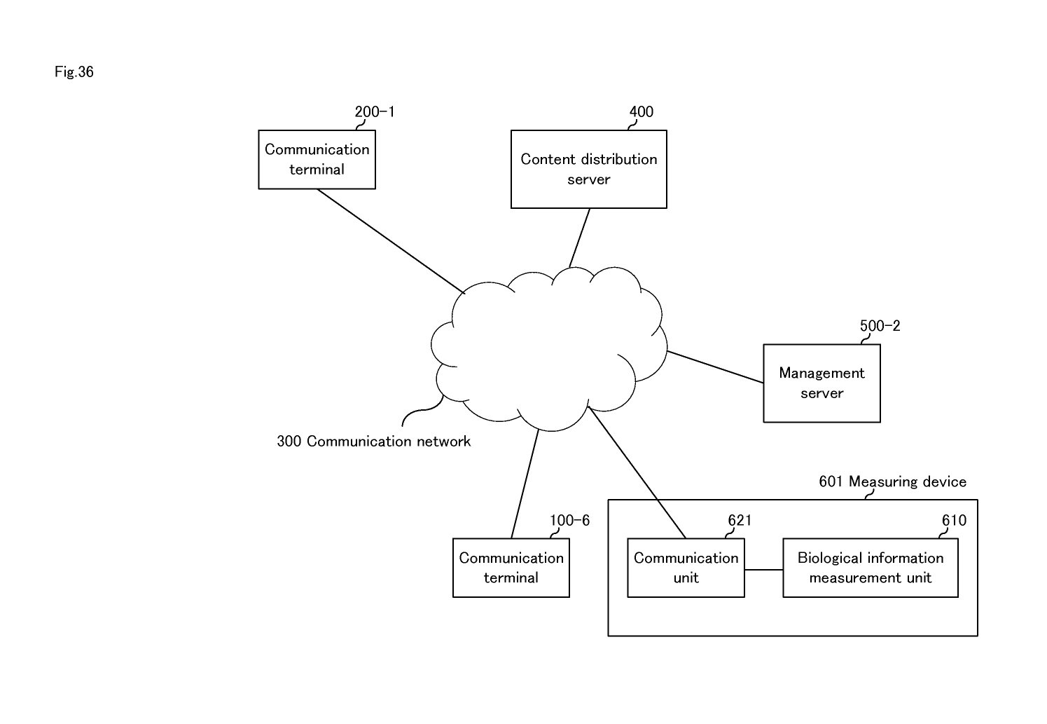

FIG. 36 shows an information processing system where biological information measurement unit 110 disposed in communication terminal 100-3 according to the fifth embodiment is disposed in measuring device 601 separated as biological information measurement unit 610 into communication unit 621 and communication terminal 100-6 according to the tenth embodiment of the present invention.

Communication terminal 200-1 and content distribution server 400 are similar to those shown in FIG. 27.

Measuring device 601, which has a structure that is attachable to a viewer, transmits the biological information of the viewer measured by biological information measurement unit 610 to management server 500-2 via communication network 300 by using communication unit 621. For example, measuring device 600 has a seal structure that is attachable to the vicinity of a breast or a structure such as a thermometer type, a wristband type, or a wristwatch type. When communication unit 621 transmits the biological information to communication network 300, the biological information can be transmitted via communication unit 140 of communication terminal 100-6.

Communication terminal 100-6 is configured by removing biological information measurement unit 110 and biological information acquisition unit 121 from the components of communication terminal 100-3 according to the fifth embodiment.

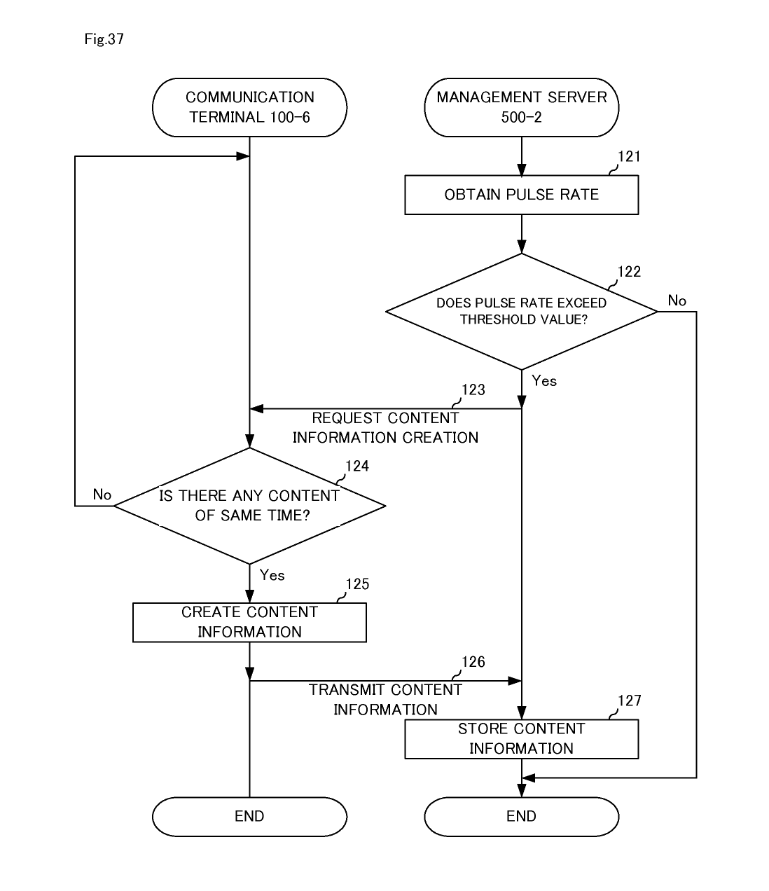

Hereinafter, an information processing method in the embodiment shown in FIG. 36 will be described. First, the processing to create content information at communication terminal 100-6 and management server 500-2 shown in FIG. 36 will be described referring to FIG. 37. An example where biological information is a pulse rate will be described. An example where output unit 150 outputs content by displaying the content will be described.

First, content information creation unit 162 writes contents that are being displayed by output unit 150 in storage unit 182 in association with the output time. For the writing timing, a predetermined period or time can be used. Storage unit 182 deletes the contents after a predetermined time has elapsed from the writing of the contents. The contents written in storage unit 182 by content information creation unit 162 indicate the contents that are being displayed by output unit 150, or the contents themselves. Any information can be used as long as it enables creation of content information.

Biological information measurement unit 610 of measuring device 601 attached to a viewer who is operating communication terminal 100-6 measures the pulse rate of the viewer at predetermined timing. Then, biological information measurement unit 610 transmits the measured pulse rate together with the measurement time of the pulse rate to management server 500-2 via communication network 300 by using communication unit 621. After communication unit 510 of management server 500-2 has received the pulse rate transmitted from measuring device 601, in step 121, condition determination unit 540 acquires the pulse rate received by communication unit 510. At this time, condition determination unit 540 also acquires the measurement time received simultaneously with the pulse rate.

In step 122, condition determination unit 540 determines whether the acquired pulse rate exceeds a threshold value preset in the threshold value table of storage unit 531. When condition determination unit 540 determines that the acquired pulse rate exceeds the threshold value preset in the threshold value table of storage unit 531, in step 123, condition determination unit 540 makes a request to create content information to communication terminal 100-6 via communication network 300 by using communication unit 510. At this time, condition determination unit 540 also transmits the measurement time received together with the pulse rate to communication terminal 100-6. On the other hand, when condition determination unit 540 determines that the pulse rate received by communication unit 510 does not exceed the threshold value preset in the threshold value table of storage unit 531, the processing is ended.

Then, after communication unit 140 of communication terminal 100-6 has received the request to create content information from management server 500-2, in step 124, content information creation unit 162 determines whether the contents of the output time similar to the measurement time received together with the request have been stored in storage unit 182.

When content information creation unit 162 determines that the contents of the output time similar to the measurement time have been stored in storage unit 182, content information creation unit 162 reads the contents from storage unit 182. Then, in step 125, content information creation unit 162 creates content information based on the read contents to output the content information to content information providing unit 171. In step 126, content information providing unit 171 transmits the content information output from content information creation unit 162 to management server 500-2 via communication network 300 by using communication unit 140.

After communication unit 510 of management server 500-2 has received the content information transmitted from communication terminal 100-6, in step 127, control unit 520 writes and stores the content information received by communication unit 510 in storage unit 531.

On the other hand, when content information creation unit 162 determines that the contents of the output time similar to the measurement time have not been stored in storage unit 182 in step 124, content information creation unit 162 waits for transmission of the request to create content information from management server 500-2.

Depending on the measurement timing of the biological information and the output timing of the contents, the measurement time and the output time do not necessarily match each other. Accordingly, by securing a certain margin, content information creation unit 162 can search for the content of the output time that is included in a period of several seconds or several minutes before or after the measurement time transmitted from communication terminal 200-3 in storage unit 182.

Processing for outputting the content information after the content information has been stored in storage unit 531 of management server 500-2 is similar to that of the fifth embodiment.

As described above, according to the present invention, the biological information of the viewer is measured and, when the measured biological information satisfies the preset conditions, the contents viewed by the viewer are notified to the checker. Thus, the checker can know that information inappropriate to the viewer has been output.

Communication terminals 100-1 to 100-6 and 200-1 to 200-3, which are electronic devices such as mobile phones, mobile terminals, tablet or notebook PCs (Personal Computers), smartphones, PDAs (Personal Digital Assistants), game machines, digital cameras, or electronic books, have content output functions and communication functions.

The processing performed by each component included in each of communication terminals 100-1 to 100-6 and 200-1 to 200-3 and management servers 500-1 and 500-2 can be executed by a logical circuit created according to each purpose. A computer program (hereinafter, referred to as program) describing processing contents as procedures can be recorded in a recording medium readable by each of communication terminals 100-1 to 100-6 and 200-1 to 200-3 and management servers 500-1 and 500-2. Then, the program recorded in the recording medium can be read by each of communication terminals 100-1 to 100-6 and 200-1 to 200-3 and management servers 500-1 and 500-2 to be executed. The recording medium readable by each of communication terminals 100-1 to 100-6 and 200-1 to 200-3 and management servers 500-1 and 500-2 is a movable recording medium such as a floppy (registered trademark) disk, a magneto-optical disk, a DVD, or a CD, or a memory such as a ROM or a RAM, or a HDD included in each of communication terminals 100-1 to 100-6 and 200-1 to 200-3 and management servers 500-1 and 500-2. The program recorded in the recording medium is read by a CPU (not shown) disposed in each of communication terminals 100-1 to 100-6 and 200-1 to 200-3 and management servers 500-1 and 500-2, and the same processing as that described above is performed under CPU control. In this case, the CPU operates as a computer that executes the program read from the recording medium where the program has been recorded.

Some or all of the aforementioned embodiments can be described as in appendixes below, but not limited to these.

[Appendix 1]

An information processing system including:

a biological information measurement unit that measures biological information;

a first information processing terminal that includes a first output unit configured to output information, and a biological information acquisition unit configured to acquire the biological information measured by the biological information measurement unit from the biological information measurement unit;

a second information processing terminal; and

a condition determination unit that determines whether or not the biological information acquired by the biological information acquisition unit satisfies preset conditions.

In this case, the first information processing terminal includes a content information creation unit configured to create, when the condition determination unit determines that the biological information satisfies the conditions, content information indicating contents of the information output from the first output unit; and

a content information providing unit that provides the content information created by the content information creation unit to the second information processing terminal, wherein:

the second information processing terminal includes a second output unit configured to output information indicated by the content information provided from the content information providing unit.

[Appendix 2]

The information processing system according to Appendix 1, characterized in that:

the biological information measurement unit measures, when a pulse rate or a heart rate of a user who is operating the first information processing terminal is used as the biological information, the biological information with a predetermined short period or a predetermined rate shorter than a measuring period of a pulse rate or a heart rate used for determination; and

the biological information acquisition unit predicts the biological information in the measuring period used for the determination from the biological information acquired from the biological information measurement unit.

[Appendix 3]

The information processing system according to Appendix 1 or 2, characterized in that the condition determination unit determines that the biological information satisfies the conditions when the biological information exceeds a preset threshold value.

[Appendix 4]

The information processing system according to Appendix 1 or 2, characterized in that the condition determination unit determines that the biological information satisfies the conditions when a state where the biological information exceeds a preset threshold value continues for a predetermined time or more.

[Appendix 5]

The information processing system according to any one of Appendixes 1 to 4, further including a storage unit that stores the content information created by the content information creation unit, characterized in that the content information providing unit reads, after a request for the content information has been received from the second information processing terminal, the content information stored in the storage unit to provide the information to the second information processing terminal.

[Appendix 6]

The information processing system according to any one of Appendixes 1 to 4, characterized in that:

the second information processing terminal includes a storage unit configured to store the content information provided from the content information providing unit; and

the second output unit reads, after a request to output content information has been received from outside the second information processing terminal, the content information stored in the storage unit to output the information.

[Appendix 7]

The information processing system according to any one of Appendixes 1 to 6, characterized in that the first information processing terminal includes at least one of the biological information measurement unit and the condition determination unit.

[Appendix 8]

The information processing system according to any one of Appendixes 1 to 7, characterized in that the first output unit displays the information.

[Appendix 9]

The information processing system according to any one of Appendixes 1 to 8, characterized in that the first output unit outputs the information as a voice.

[Appendix 10]

An information processing terminal including:

an output unit that outputs information;

a biological information acquisition unit that acquires biological information;

a condition determination unit that determines whether or not the biological information acquired by the biological information acquisition unit satisfies preset conditions;

a content information creation unit that creates, when the condition determination unit determines that the biological information satisfies the conditions, content information indicating contents of the information output from the output unit; and

a content information providing unit that provides the content information created by the content information creation unit.

[Appendix 11]

The information processing terminal according to Appendix 10, further including:

a casing that includes the output unit, the biological information acquisition unit, the condition determination unit, the content information creation unit, and the content information providing unit; and

an attaching unit added to the casing to attach the casing to a user,

the information processing terminal being a wristwatch type or a wristband type that includes a biological information measurement unit for measuring the biological information disposed on the casing side or the attaching unit's side that is next to the skin of the user.

[Appendix 12]

An information processing method including:

processing for outputting information;

processing for acquiring biological information;

processing for determining whether or not the acquired biological information satisfies preset conditions;

processing for creating, when the biological information satisfies the conditions, content information indicating contents of the output information; and

processing for providing the created content information.

[Appendix 13]

A program for causing a computer to execute:

a procedure for outputting information;

a procedure for acquiring biological information;

a procedure for determining whether the acquired biological information satisfies preset conditions;

a procedure for creating, when the biological information satisfies the conditions, content information indicating contents of the output information; and

a procedure for providing the created content information.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

1. An information processing system comprising:
a biological information measurement unit that measures biological information;
a first information processing terminal that includes a first output unit configured to output information, and a biological information acquisition unit configured to acquire the biological information measured by the biological information measurement unit from the biological information measurement unit;
a second information processing terminal; and
a condition determination unit that determines whether or not the biological information acquired by the biological information acquisition unit satisfies preset conditions, wherein:
the first information processing terminal includes a content information creation unit configured to create, when the condition determination unit determines that the biological information satisfies the conditions, content information indicating contents of the information output from the first output unit; and
a content information providing unit that provides the content information created by the content information creation unit to the second information processing terminal, wherein:
the second information processing terminal includes a second output unit configured to output information indicated by the content information provided from the content information providing unit.

2. The information processing system according to claim 1, wherein:
the biological information measurement unit measures, when a pulse rate or a heart rate of a user who is operating the first information processing terminal is used as the biological information, the biological information with a predetermined short period or a predetermined rate shorter than a measuring period of a pulse rate or a heart rate used for determination; and
the biological information acquisition unit predicts the biological information in the measuring period used for the determination from the biological information acquired from the biological information measurement unit.

3. The information processing system according to claim 1, wherein the condition determination unit determines that the biological information satisfies the conditions when the biological information exceeds a preset threshold value.

4. The information processing system according to claim 1, wherein the condition determination unit determines that the biological information satisfies the conditions when a state where the biological information exceeds a preset threshold value continues for a predetermined time or more.

5. The information processing system according to claim 1, further comprising a storage unit that stores the content information created by the content information creation unit,
wherein the content information providing unit reads, after a request for the content information has been received from the second information processing terminal, the content information stored in the storage unit to provide the information to the second information processing terminal.

6. The information processing system according to claim 1, wherein:
the second information processing terminal includes a storage unit configured to store the content information provided from the content information providing unit; and
the second output unit reads, after a request to output content information has been received from outside the second information processing terminal, the content information stored in the storage unit to output the information.

7. The information processing system according to claim 1, wherein the first information processing terminal includes at least one of the biological information measurement unit and the condition determination unit.

8. The information processing system according to claim 1, wherein the first output unit displays the information.

9. The information processing system according to claim 1, wherein the first output unit outputs the information as a voice.

10. An information processing terminal comprising:
an output unit that outputs information;
a biological information acquisition unit that acquires biological information;
a condition determination unit that determines whether or not the biological information acquired by the biological information acquisition unit satisfies preset conditions;
a content information creation unit that creates, when the condition determination unit determines that the biological information satisfies the conditions, content information indicating contents of the information output from the output unit; and
a content information providing unit that provides the content information created by the content information creation unit.

11. The information processing terminal according to claim 10, further comprising:
a casing that includes the output unit, the biological information acquisition unit, the condition determination unit, the content information creation unit, and the content information providing unit; and
an attaching unit added to the casing to attach the casing to a user,
the information processing terminal being a wristwatch type or a wristband type that includes a biological information measurement unit for measuring the biological information disposed on the casing side or the attaching unit's side that is next to the skin of the user.

12. An information processing method comprising:
processing for outputting information;
processing for acquiring biological information;
processing for determining whether or not the acquired biological information satisfies preset conditions;
processing for creating, when the biological information satisfies the conditions, content information indicating contents of the output information; and
processing for providing the created content information.
